(19) United States
(12) Patent Application Publication
(10) Pub. No.: US 2026/0185982 A1
(43) Pub. Date: Jul. 2, 2026

(54) IMPROVED MONOCYTE ACTIVATION TESTS USING HUMAN PLATELET SERUM

(71) Applicant: MAT RESEARCH B.V., Leiden (NL)

(72) Inventors: Koen Abraham MARIJT, Oegstgeest (NL); Guillermo FREIRE SANCHEZ, Leiden (NL)

(73) Assignee: MAT RESEARCH B.V., Leiden (NL)

(21) Appl. No.: 19/131,548

(22) PCT Filed: Nov. 22, 2023
(86) PCT No.: PCT/EP2023/082633

§ 371 Date: May 21, 2025

(30) Foreign Application Priority Data
Nov. 22, 2022 (EP) 22208857.7

(51) Int. Cl. G01N 33/50; G01N 33/68

(52) U.S. Cl. CPC G01N 33/5047; G01N 33/6869; G01N 2333/5412

(57) ABSTRACT

The present invention is in the field of in vitro assays, particularly in the field of pyrogen and endotoxin detection. The invention provides improved compositions suitable for use in animal-free testing such as for use in a monocyte activation test. The improved compositions allow for test responses with increased sensitivity, improved dynamic range, and improved goodness of fit.

### FIELD OF THE INVENTION

The present invention is in the field of in vitro assays, particularly in the field of pyrogen and endotoxin detection. The invention provides improved compositions suitable for use in animal-free testing such as for use in a monocyte activation test. The improved compositions allow for test responses with increased sensitivity, reduced variability, improved dynamic range, and improved goodness of fit.

### BACKGROUND OF THE INVENTION

Detecting the presence of pyrogens and endotoxins is highly relevant in quality and safety testing of pharmaceutical compositions and medical devices due to the capacity of pyrogens and endotoxins for causing severe adverse reactions in patients. Traditionally used detection assays include the rabbit pyrogen test (RPT) and the limulus amebocyte lysate assay (LAL), also known as bacterial endotoxin test (BET). These tests are costly, time consuming, and require the use of test animals. Further, the limulus amebocyte lysate assay is generally limited to detection of only Gram-negative bacteria and is prone to false-positives.

An alternative to the above assays is the monocyte activation test (MAT), which does not require the use of animals and is more representative of the human immune response. The MAT is opted to replace the Rabbit Pyrogen Test (RPT) by the year 2025 by the European commission. It is a test which is governed by specific regulatory guidelines for sample preparation, testing, and analysis of results, established in the European Pharmacopoeia (Ph. Eur.; European Pharmacopoeia 10^{th} Edition, 2019, Monograph 2.6.30, the Council of Europe). Constrained by these guidelines, the currently used protocols have low throughput, are costly, and require the use of large amount of reagents and sample volumes.

To date, the classical MAT is typically performed using the medium supplements Fetal bovine serum (FBS) or human AB (hAB) serum (Ph. Eur.; European Pharmacopoeia, supra). However, while FBS provides good reactivity towards endotoxin detection, it poorly detects low concentrations of non-endotoxin pyrogens. Conversely, hAB serum has been shown to provide good sensitivity towards non-endotoxin pyrogens, but has a reduced ability to detect endotoxins, for instance as compared to FBS. Therefore, a MAT assay using fetal bovine serum and one using hAB serum are required to detect the full spectrum of pyrogens with adequately high sensitivity (Molenaar-de Backer 2021, ALTEX—Alternatives to animal experimentation, 38(2); 307-315). Accordingly, there is still a need for improved pyrogen and endotoxin detection assays which are animal-free. Further, there is still a need for improved monocyte activation tests. There is a need for improving the sensitivity of monocyte activation tests. There is a need for reducing the costs of monocyte activation tests. There is a need for improving reliability of monocyte activation tests. There is a need for simplifying monocyte activation tests. There is a need for reducing the need to perform multiple parallel tests.

### SUMMARY OF THE INVENTION

The inventors have found that human platelet lysate (hPL) is a human based medium supplement that results in assays showing surprisingly high sensitivity to both endotoxin and multiple non-endotoxin pyrogens, thus showing advantages over other medium supplements. For instance, as shown in the Examples, it was found that assays using either one of the commonly used supplements hAB serum and FBS were unable to effectively detect both endotoxins and non-endotoxin pyrogens at low concentrations. However, assays using hPL showed consistently strong sensitivity towards both endotoxins and non-endotoxin pyrogens, particularly as compared to FBS and hAB.

Accordingly, in a first aspect, there is provided a method for detecting a pyrogen in a sample, the method comprising the steps of:
i) providing one or more samples;
ii) contacting the sample with peripheral blood mononuclear cells (PBMCs) in an incubation medium comprising human platelet lysate (hPL); and
iii) determining the response of the PBMCs.

In some embodiments, the method is a monocyte activation test. In some embodiments, the incubation medium has a volume of at most 300 μL or at most 250 μL per sample, preferably of from 20 to 250 μL, more preferably of from 30 to 175 μL, most preferably of from 50 to 110 μL In some embodiments, the contacting of step ii) is performed in a standardised 96-well plate or 384-well plate, preferably a 96-well plate. In some embodiments, the incubation medium comprises from 0.05 to 20 vol. % human platelet lysate, preferably from 0.5 to 4 vol. %, more preferably from 0.8 to 3 vol. %, even more preferably from 1 to 2.5 vol. %, most preferably from 1.2 to 2.2 vol. %, such as about 2 vol. %. In some embodiments, the response of the PBMCs that is determined is the excretion of an inflammatory cytokine such as IL-6, IL-1 beta, IL-8, TNF-alpha, MCP-1, IFN-alpha, IFN-beta, IFN-gamma, IFN-lambda, of a prostaglandin, or of a high-mobility-group-protein. In some embodiments, the response of the PBMCs is higher than the response of PBMCs in a comparative method that differs only in the replacement of human platelet serum by human AB serum or by fetal bovine serum, or in the absence of human platelet serum. In some embodiments, the response of the PBMCs is determined by an ELISA assay. In some embodiments, the PBMCs are present at a density of at most 500×1000 cells/cm², preferably at most 250×1000 cells cm². In some embodiments, the PBMCs are present at a density of from about 10×1000 cells/cm² to about 350×1000 cells/cm² optionally to about 300×1000 cells/cm², preferably of from about 50×1000 cells/cm² to about 150×1000 cells/cm², more preferably of from about 90×1000 cells/cm² to about 130×1000 cells/cm². In some embodiments, the limit of quantitation of lipopolysaccharides is below 0.01 EEU/mL, and/or the limit of quantitation of triacylated lipopeptide is below 0.1 ng/mL, and/or the limit of quantitation of bacterial protein is below 1, preferably below 0.5 ng/mL. In some embodiments, in step iii) the determined response of the PBMCs for a plurality of identical samples has a coefficient of variation of at most 30%. In some embodiments, the incubation medium has a volume of from about 80 to about 120 μL, and the PBMCs are present at a density of from about 90 to about 130×1000 cells/cm². In a further aspect, there is provided a method for releasing a pharmaceutical composition or a medical device for use, the method comprising subjecting the pharmaceutical composition or a sample derived from the medical device to a method of the first aspect. In a further aspect, there is provided a kit of parts comprising a pyrogen or endotoxin standard and a vial comprising human platelet lysate, further optionally comprising PBMCs.

### DESCRIPTION OF THE INVENTION

In an aspect, there is provided a method for detecting a pyrogen in a sample, the method comprising the steps of:
i) providing one or more samples;
ii) contacting the sample with peripheral blood mononuclear cells (PBMCs) in an incubation medium comprising human platelet lysate (hPL); and
iii) determining the response of the PBMCs.

This method is attractive because it has advantages compared to methods currently available in the art: the invention has no requirement for the use of test animals, has improved sensitivity towards endotoxin and/or non-endotoxin pyrogens, has improved range of response towards endotoxin and/or non-endotoxin pyrogens, has improved reliability of PBMC response determination, has low variability between measurements, and thus leads to decreased costs. The steps of the method are preferably performed in numerical order.

### Pyrogens

Pyrogens are well-known in the art. They are substances that can trigger an immune response in a subject by activating a cascade of immunological processes, typically characterized by an increase in the body's internal temperature outside of normal levels (fever). A biological activity of a pyrogen is its capacity to produce fever in a subject, alternatively referred to herein as its pyrogenicity. A pyrogen may be an exogenous pyrogen. “Exogenous” or “external” pyrogens refer to pyrogens originating outside of the subject's body. A pyrogen may be an endotoxin, preferably a lipopolysaccharide (LPS). Endotoxins, such as lipopolysaccharides, are cellular components of bacteria such as Gram-negative bacteria which constitute the main component of their outer cell walls. The presence of endotoxins in the blood stream of a subject is associated with multiple adverse symptoms, including fever, hypotension, nausea, shivering, and shock, and can lead to complications such as disseminated intravascular coagulation (DIC), endotoxin shock, and acute respiratory distress syndrome (ARDS). A pyrogen may be a non-endotoxin pyrogen (NEP). Non-endotoxin pyrogens include microbe-associated molecular patterns (MAMPs) and pathogen-associated molecular patterns (PAMPs), with examples being bacterial cellular components such as bacterial proteins (e.g., flagellins), peptidoglycans, lipoproteins, lipoteichoic acid, fibroblast-stimulating lipopeptide 1, macrophage-activating lipopeptide-2, viral pyrogens, yeast pyrogens, and fungal pyrogens (e.g., yeast or fungal polysaccharides). In some cases, a flagellin may be from a Gram-positive bacterium, for example from Bacillus subtilis.

A pyrogen may be a product- or a process-related impurity that is present in a pharmaceutical composition or on a surface (for example of a medical device). Examples of pyrogenic impurities are chemical agents, for example polyadenylic acid, polyuridylic acid, polybionosinic acid, dinitrophenol, trinitrophenol, 4,6-dinitro-o-cresol, N-phenyl-P-naphthylamine, aldo-a-napththylamine, metals, and nanoparticles (typically <1 nm), and any other impurity that displays pyrogenicity.

A pyrogen may be a damage-associated molecular pattern (DAMP), which refers to biomolecules that are typically released by dying or damaged cells. Examples of DAMP pyrogens include byglycan, decorin, versican, hyaluronoan, fibronectin, tenascin, uric acid, S100 proteins, ATP, GTP, F-actin, cyclophilin A, histones, HMGB1, HMGN1, IL-1a, IL-33, SAP130, DNA, RNA, mtDNA, TFAM, formyl peptide, mROS, calreticulin, defensins, heat shock proteins, and any other biomolecule released by a cell that displays pyrogenicity. A pyrogen may be a toll-like receptor 1/2 (TLR1/2) agonist. A pyrogen may be a medium component of a pharmaceutical composition. Examples of such components include excipients, solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and the like. A pyrogen may be a triacylated lipopeptide. A pyrogen may be an endogenous pyrogen. “Endogenous” or “internal” pyrogens refer to pyrogens produced by the body of a subject following contacting with an exogenous pathogen. An endogenous pyrogen may be associated with an inflammatory response. An endogenous pyrogen may be a DAMP. An endogenous pyrogen may be a toll-like receptor 1/2 (TLR1/2) agonist. Examples of endogenous pyrogens include cytokines and chemokines.

In some embodiments, the pyrogen is an exogenous pyrogen. In some embodiments, the pyrogen is an endotoxin. In some embodiments, the endotoxin is a cellular component, preferably a lipopolysaccharide, of a Gram-negative bacterium. “Gram-negative” bacterium refers to a bacterium that generally does not retain the crystal violet stain used in the standard Gram staining method, as opposed to a “Gram-positive” bacterium, which generally retains the stain. In some embodiments, the Gram-negative bacterium is a pathogenic or potentially pathogenic bacterium. Examples of pathogenic or potentially pathogenic Gram-negative bacteria are bacteria of the genera Escherichia, Salmonella, Shigella, Pseudomonas, Neisseria, Haemophilus, Bordetella, Vibrio, and the like. In some embodiments, the pyrogen is a non-endotoxin pyrogen (NEP). In some embodiments, the pyrogen is a cellular component, preferably a bacterial protein (e.g., flagellin), of a Gram-positive bacterium. In some embodiments, the Gram-positive bacterium is pathogenic or potentially pathogenic. Examples of pathogenic or potentially pathogenic Gram-positive bacteria are bacteria of the genera Streptococcus, Staphylococcus, Corynebacterium, Listeria, Bacillus (e.g., Bacillus subtilis), Clostridium, and the like. In some embodiments, the pyrogen is a product- or a process-related impurity that is present in a pharmaceutical composition or on a surface. In some embodiments, the pyrogen is a damage-associated molecular pattern (DAMP). In some embodiments, the pyrogen is a medium component of a pharmaceutical composition. In some embodiments, the pyrogen is a toll-like receptor 1/2 (TLR1/2) agonist. In some embodiments, the pyrogen is a triacylated lipopeptide. In some embodiments, the pyrogen is an endogenous pyrogen. In some embodiments, the endogenous pyrogen is associated with an inflammatory response. In some embodiments, the endogenous pyrogen is a damage-associated molecular pattern (DAMP). In some embodiments, the endogenous pyrogen is a cytokine. In some embodiments, the endogenous pyrogen is a chemokine. In some embodiments, the endogenous pyrogen is a toll-like receptor 1/2 (TLR1/2) agonist.

### Step i) Provision of Samples

In step i) of the method, one or more samples are provided. A sample can be taken (derived) from an original source, for example from a product such as a pharmaceutical composition or a medical device. A sample can also be a subsample which is taken from an original sample (or from another subsample). A sample can also be, or taken from, a subsample that arises from dilution or concentration of an original sample.

A sample can also be a replicate of an original sample or of a subsample. Replicate samples are preferably intended to be identical, more preferably they are identical. In cases where a sample is a replicate of an original sample or of a subsample, at least two, at least three, or at least four, preferably at least four, replicates of the original sample or of the subsample are provided. Replicates may, for example, be prepared individually or may arise from taking equal subsamples from an original sample or of a subsample. A sample is preferably a liquid sample, more preferably an aqueous sample.

A sample may be taken from a pharmaceutical composition to be tested for the presence of a pyrogen, for example from a therapeutic composition, diagnostic composition, or a composition for preventing a disease or condition or reducing the symptoms thereof (such as a vaccine). A pharmaceutical composition may be in any form. In some embodiments, the pharmaceutical composition is a vaccine.

A sample may be taken from a surface to be tested for the presence of a pyrogen, for example from a surface of a medical device or instrument. Examples of medical devices and instruments include bedpans, cannulas, cardioverters, defibrillators, catheters, dialysers, electrocardiograph machines, enema equipment, endoscopes, gas cylinders, gauze sponges, surgical scissors, hypodermic needles, syringes, infection control equipment such as masks, gowns, face shields, and goggles, instrument sterilizers, kidney dishes, nasogastric tubes, surgical scalpels, nebulizers, ophthalmoscopes, otoscopes, pipettes, proctoscopes, radiographers, sphygmomanometers, thermometers, tongue depressors, transfusion kits, tuning forks, ventilators, watches, and the like. Such a sample may be taken, for example, by rinsing the surface to be tested with a solution (e.g., water or a buffer), collecting the rinsate, and utilizing it for sample preparation.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 132, at least 164, at least 196, at least 228, at least 260, at least 292, at least 324, at least 356, or at least 384 samples are provided. In some embodiments, at least 50, preferably at least 97 samples are provided. In some embodiments, at least 96 samples are provided. In some embodiments, at least 384 samples are provided. Advantageously, the method according to the invention can be practiced using a 384-well plate.

In some embodiments, in step i) a further control sample is provided. A control sample can comprise a pyrogen standard (positive control) or can be free of pyrogens (negative control). Inclusion of pyrogen-positive control samples of known pyrogen concentrations can enable increased quantification accuracy. For example, using multiple samples comprising different pyrogen concentrations, a standard curve may be prepared. A control sample can be a sample from a (reference) standard. Such standards are described later herein.

In some embodiments, a control sample is an endotoxin sample, preferably a lipopolysaccharide (LPS) sample. Endotoxin is typically measured in endotoxin units per mL (EEU/mL or EU/mL, where EEU is ‘equivalent endotoxin units’). One EEU/mL (EU/mL) is equal to approximately 0.1-0.2 ng endotoxin/mL of solution, preferably 0.15 ng/mL. In embodiments wherein the control sample is an endotoxin, preferably a lipopolysaccharide, sample, the control sample preferably comprises from about 0.005 to about 15 endotoxin units per mL. In some embodiments, the control sample comprises from about 0.005 to about 1 endotoxin units per mL. In some embodiments, the control sample comprises from about 0.008 to about 0.5 endotoxin units per mL, or from about 0.01 to about 0.4, preferably from about 0.05 to about 0.3, more preferably from about 0.1 to about 0.2 EU/mL.

In some embodiments, a control sample is a non-endotoxin pyrogen (NEP) sample, for example a cellular component such as a bacterial protein (e.g., a flagellin). In some embodiments, a control sample is a triacylated lipopeptide. In some embodiments, a control sample is a toll-like receptor 1/2 (TLR1/2) agonist. In some embodiments, a toll-like receptor 1/2 (TLR1/2) agonist in a non-endotoxin control sample is a synthetic molecule. Such molecules are commercially available, an example being PAM3CSK4 (CAS No.: 112208-00-1). In some embodiments wherein the control sample is a non-endotoxin pyrogen sample, the control sample preferably comprises from about 0.01 to about 125 ng/mL non-endotoxin pyrogen. In some embodiments, the control sample comprises from about 0.1 to about 20 ng/mL non-endotoxin pyrogen, preferably from about 0.1 to about 15 ng/mL, more preferably from about 1 to about 10 ng/mL.

Step ii) Contacting with PBMCs

In step ii), the sample or samples are contacted with peripheral blood mononuclear cells (PBMCs) in an incubation medium comprising human platelet lysate (hPL). In some embodiments, contacting is done with whole peripheral blood, or with a fraction thereof, containing PBMCs. In some embodiments, contacting is done with isolated PBMCs. Blood fractions containing PBMCs and isolated PBMCs may be obtained with standard methods, for example using leukapheresis and/or density-gradient centrifugation.

In some embodiments, contacting is done with a PBMC cell line. In some embodiments, contacting is done with immortal or non-immortal cells, preferably with non-immortal cells. In some embodiments, contacting is done with PBMCs obtained from a single donor. In some embodiments, contacting is done with PBMCs obtained from pooled whole peripheral blood from multiple donors. In cases wherein PBMCs are obtained from a single or multiple donors, the donors are preferably qualified according to standardized guidelines, more preferably as described in Sections 5-3, 5-4, 5-5, 6-3, and/or in Monograph 2.6.30 of the European Pharmacopoeia (Ph. Eur.; European Pharmacopoeia 10th Edition, 2019, the Council of Europe).

In some embodiments, contacting is done with fresh PBMCs. In preferred embodiments, contacting is done with cryo-preserved PBMCs. Cryopreservation of PBMCs may be done according to standard procedures, for example as described in standard handbooks such as Hubel, A., 2018: Preservation of Cells: A Practical Manual, 1st Edition, Wiley-Blackwell, NJ, USA. In some embodiments, the PBMCs are mammalian, preferably human. In some embodiments, the PBMCs are leukocytes. In some embodiments, the PBMCs are macrophages. In preferred embodiments, the PBMCs comprise or are monocytes, preferably mammalian monocytes, more preferably human monocytes. In some embodiments, the macrophages or monocytes are derived from pluripotent stem cells. PBMCs are well known and well characterized, and generally comprise 10-20% monocytes. Preferred PBMCs are non-immortal PBMCs, preferably non-immortal monocytes.

The method is preferably a monocyte activation test, or part of a monocyte activation test. More preferably, the monocyte activation test is according to the guidelines laid out in Monograph 2.6.30 of the European Pharmacopoeia (supra).

Contacting with the PBMCs in step ii) can be addition of the sample or samples to the medium comprising the PBMCs. It can also be addition of the medium comprising the PBMCs to the sample or samples. It may be done in any suitable receptacle, for example a microplate (with one or more wells), a tube (such as an Eppendorf tube), a flask (such as an Erlenmeyer flask), a bottle (such as a Schott bottle), a fermenter, and the like. In preferred embodiments, contacting is done in a standardized 96-well plate. In some embodiments, contacting is done in a standardized 384-well plate. Standardized well plates are widely available from commercial suppliers. Suitable standards are ANSI/SLAS standards, preferably all five of 1-2004 (R2012), 2-2004 (R2012), 3-2004 (R2012), 4-2004 (R2012), and 6-2012 (R2012).

In cases where a 96-well plate or a 384-well plate is used, a single sample can be placed in each well. The invention advantageously allows the use of 384-wells plates. Use of 384-well plates has the further advantages of enabling higher detection throughput (as more samples can be tested simultaneously) and decreasing reagent requirements and overall costs. In standardized use of well-plates for MAT, a given amount of wells is required for control or reference samples. This limits the amount of wells that are available for actual test samples. A 384-well plate has a better test-to-control ratio because after allocation of wells to the required control sample, more wells remain available for test samples. In this context, the following can be a conventional allocation of wells for a 96-well plate, using 4 replicates per data point:

| | | Wells for |
|---|---|---|
| | Samples | quadruplicate |
| LPS curve | 8 | 32 |
| Test sample 1 in three concentrations | 3 | 12 |
| Test sample 1 LPS spike | 3 | 12 |
| Test sample 1 test sample 1 NEP control | 2 | 8 |
| Test sample 2 in three concentrations | 3 | 12 |
| Test sample 2 LPS spike | 3 | 12 |
| Test sample 2 test sample 1 NEP control | 2 | 8 |
| TOTAL WELLS | | 96 |

Alternately, with 2 dilutions per test sample and omitting the NEP control, the following can apply:

| | | Wells for |
|---|---|---|
| | Samples | quadruplicate |
| LPS curve | 8 | 32 |
| Test sample 1 in two concentrations | 2 | 8 |
| Test sample 1 LPS spike | 2 | 8 |
| Test sample 2 in two concentrations | 2 | 8 |
| Test sample 2 LPS spike | 2 | 8 |
| Test sample 3 in two concentrations | 2 | 8 |
| Test sample 3 LPS spike | 2 | 8 |
| Test sample 4 in two concentrations | 2 | 8 |
| Test sample 4 LPS spike | 2 | 8 |
| TOTAL WELLS | | 96 |

It should be noted that a sample of a product to be analysed can lead to multiple samples that are provided in the method for detecting a pyrogen. For instance, in the above table, a single test sample 1 results in a plurality of samples when each well is seen as comprising a provided sample. This difference will be clear from context when not explicitly indicated.

The PBMCs, preferably comprising monocytes, may be present at a specific density during the contacting step, preferably measured in cells/cm² (cm² refers to the growing area, which is preferably the area that is the cross-section of a well; preferred wells are flat-bottomed wells). The density of the PBMCs in the context of the disclosure refers to the PBMC density used per contacted sample. The skilled person can easily calculate the density in cells/cm² in a receptacle, preferably a well of a standardized 96-well or 384-well plate, taking into account the growing area (cm²), the cell concentration (cells/mL) and the volume (mL), for instance by using a cell counter or by using microscopic techniques. This density is a commonly used parameter and a skilled person understands that some dead cells may be present in a population. In some embodiments, the PBMCs, preferably comprising monocytes, are present at a density of at most 500×1000 cells/cm², preferably at most 250×1000 cm².

In some embodiments, the PBMCs, preferably comprising monocytes, are present at a density of from about 10×1000 cells/cm² to about 350×1000 cells/cm², preferably of from about 50×1000 cells/cm² to about 150×1000 cells/cm², preferably of from about 60×1000 cells/cm² to about 140×1000 cells/cm², preferably of from about 70×1000 cells/cm² to about 130×1000 cells/cm², preferably of from about 80×1000 cells/cm² to about 120×1000 cells/cm², more preferably of from about 90×1000 cells/cm² to about 130×1000 cells/cm². In some embodiments, the PBMCs, preferably comprising monocytes, are present at a density of 110×1000 cells/cm² or about 110×1000 cells/cm².

The incubation medium may be have a specific volume during the contacting step, preferably measured in mL or μL. The volume of the incubation medium in the context of the disclosure refers to the volume per sample. In some embodiments, the incubation medium has a volume of at most 300 μL per sample or at most 250 μL per sample, preferably at most 250 μL per sample. In preferred embodiments, it has a volume of at most 200 μL per sample, at most 175 μL per sample, or at most 150 μL per sample, preferably of at most 250 μL or about 250 μL per sample. In some embodiments, the incubation medium has a volume of from 20 to 250 μL, preferably of from 30 to 175 μL, more preferably from 50 to 110 μL.

In some embodiments, the incubation medium has a volume of from 20 to 150 μL, preferably of from 30 to 140 μL, more preferably of from 50 to 110 μL. In some embodiments, the incubation medium has a volume of from 80 to 120 μL. In some embodiments the incubation medium has a volume of from 40 to 130 μL, preferably 60 to 120 μL, more preferably of from 70 to 115 μL, more preferably of from 75 to 105 μL, more preferably of from 85 to 105 μL.

In some embodiments, the incubation medium has a volume of from 20 to 100 μL, preferably of from 30 to 100 μL, more preferably of from 50 to 100 μL. In some embodiments, the incubation medium has a volume of from 80 to 100 μL. In some embodiments, the incubation medium has a volume of 100 μL or about 100 μL. In some embodiments, the incubation medium has a volume of from 1 to 99 μL, preferably from 30 to 70 μL such as 33 μL or 66 μL.

In some embodiments, the incubation medium has a volume of from 20 to 150 μL and the PBMCs, preferably monocytes, are present at a density of from about 10×1000 cells/cm² to about 250×1000 cells/cm². In some embodiments, the incubation medium has a volume of from 20 to 150 μL and the PBMCs, preferably monocytes, are present at a density of from about 50×1000 cells/cm² to about 150×1000 cells/cm². In some embodiments, the incubation medium has a volume of from 20 to 150 μL and the PBMCs, preferably monocytes, are present at a density of from about 90×1000 cells/cm² to about 130×1000 cells/cm².

In some embodiments, the incubation medium has a volume of from 30 to 140 μL and the PBMCs, preferably monocytes, are present at a density of from about 10×1000 cells/cm² to about 250×1000 cells/cm². In some embodiments, the incubation medium has a volume of from 30 to 140 μL and the PBMCs, preferably monocytes, are present at a density of from about 50×1000 cells/cm² to about 150×1000 cells/cm². In some embodiments, the incubation medium has a volume of from 30 to 140 μL and the PBMCs, preferably monocytes, are present at a density of from about 90×1000 cells/cm² to about 130×1000 cells/cm².

In some embodiments, the incubation medium has a volume of from 80 to 120 μL and the PBMCs, preferably monocytes, are present at a density of from about 10×1000 cells/cm² to about 250×1000 cells/cm². In some embodiments, the incubation medium has a volume of from 80 to 120 μL and the PBMCs, preferably monocytes, are present at a density of from about 50×1000 cells/cm² to about 150×1000 cells/cm². In some embodiments, the incubation medium has a volume of from 80 to 120 μL and the PBMCs, preferably monocytes, are present at a density of from about 90×1000 cells/cm² to about 130×1000 cells/cm².

In some embodiments, the incubation medium has a volume of from 50 to 110 μL and the PBMCs, preferably monocytes, are present at a density of from about 10×1000 cells/cm² to about 250×1000 cells/cm². In some embodiments, the incubation medium has a volume of from 50 to 110 μL and the PBMCs, preferably monocytes, are present at a density of from about 50×1000 cells/cm² to about 150×1000 cells/cm². In some embodiments, the incubation medium has a volume of from 50 to 110 μL and the PBMCs, preferably monocytes, are present at a density of from about 90×1000 cells/cm² to about 130×1000 cells/cm².

In some embodiments, the incubation medium has a volume of from 20 to 100 μL and the PBMCs, preferably monocytes, are present at a density of from about 10×1000 cells/cm² to about 250×1000 cells/cm². In some embodiments, the incubation medium has a volume of from 20 to 100 μL and the PBMCs, preferably monocytes, are present at a density of from about 50×1000 cells/cm² to about 150×1000 cells/cm². In some embodiments, the incubation medium has a volume of from 20 to 100 μL and the PBMCs, preferably monocytes, are present at a density of from about 90×1000 cells/cm² to about 130×1000 cells/cm².

In some embodiments, the incubation medium has a volume of from 30 to 100 μL and the PBMCs, preferably monocytes, are present at a density of from about 10×1000 cells/cm² to about 250×1000 cells/cm². In some embodiments, the incubation medium has a volume of from 30 to 100 μL and the PBMCs, preferably monocytes, are present at a density of from about 50×1000 cells/cm² to about 150×1000 cells/cm². In some embodiments, the incubation medium has a volume of from 30 to 100 μL and the PBMCs, preferably monocytes, are present at a density of from about 90×1000 cells/cm² to about 130×1000 cells/cm².

In some embodiments, the incubation medium has a volume of from 50 to 100 μL and the PBMCs, preferably monocytes, are present at a density of from about 10×1000 cells/cm² to about 250×1000 cells/cm². In some embodiments, the incubation medium has a volume of from 50 to 100 μL and the PBMCs, preferably monocytes, are present at a density of from about 50×1000 cells/cm² to about 150×1000 cells/cm². In some embodiments, the incubation medium has a volume of from 50 to 100 μL and the PBMCs, preferably monocytes, are present at a density of from about 90×1000 cells/cm² to about 130×1000 cells/cm².

In some embodiments, the incubation medium has a volume of from 80 to 100 μL and the PBMCs, preferably monocytes, are present at a density of from about 10×1000 cells/cm² to about 250×1000 cells/cm². In some embodiments, the incubation medium has a volume of from 80 to 100 μL and the PBMCs, preferably monocytes, are present at a density of from about 50×1000 cells/cm² to about 150×1000 cells/cm². In some embodiments, the incubation medium has a volume of from 80 to 100 μL and the PBMCs, preferably monocytes, are present at a density of from about 90×1000 cells/cm² to about 130×1000 cells/cm².

In some embodiments, the incubation medium has a volume of 100 μL or about 100 μL and the PBMCs, preferably monocytes, are present at a density of 110×1000 cells/cm² or about 110×1000 cells/cm². In a 96-well plate, which generally have wells having a surface area of 0.32 cm², 110×1000 cells/cm² equals about 35200 cells per well.

The incubation medium comprising the human platelet lysate (hPL) may be any medium that is suitable for PBMC, preferably mammalian PBMC, more preferably human PBMC, most preferably human monocyte culturing. Preferably, the incubation medium is according to the guidelines laid out in Monograph 2.6.30 of the European Pharmacopoeia (supra). A preferred incubation medium is RPMI (Roswell Park Memorial Institute) medium, more preferably RPMI 1640 medium, which is widely commercially available. However, other suitable media may be contemplated, for example DMEM (Dulbecco's Modified Eagle's Medium), EMEM (Eagle's Minimum Essential Medium), Ham's F-10 or F-12 medium, Iscove's Modified Dulbecco's Medium (IMDM), α-MEM (Minimum Essential Medium a), and the like, all of which are commercially available. A further example of a suitable medium is given in the Examples section later herein. In some embodiments the incubation medium is not α-MEM.

As used herein, human platelet lysate (hPL) has its common meaning in the art. It refers to a medium supplement obtained by human platelet lysis. In some embodiments, the hPL is autologous with respect to the PBMCs. In some embodiments, the hPL is allogeneic with respect to the PBMCs. Human platelet lysate may be prepared with standard methods available in the art, for example as described in Burnouf et al., 2016, Biomaterials 76; 371-387, Mohamed et al., 2020, Blood Res 55; 35-43, and Guiotto et al., 2020, J Transl Med 18:351. Human platelet lysate may be prepared from, for example, platelet concentrates prepared from fresh or stored whole blood. A platelet concentrate may be prepared using standard methods; for example, it may be prepared from anticoagulated whole blood following the buffy coat method or the PRP (platelet-rich-plasma) method, or may be prepared using plateletpheresis. Typically, the buffy coat method involves centrifugation of whole blood followed by pooling of four buffy coat units plus one plasma unit from four donors. After a second centrifugation step the platelet concentrate is filtered through a leukocyte depletion filter and stored. Typically, the PRP method involves pooling of four or five blood units followed by centrifugation to separate the blood cells from the upper layer consisting of platelets mixed with plasma. Typically, plateletpheresis involves processing of blood via an apheresis machine that uses centrifugation to remove the platelets.

hPL can be prepared from platelet concentrates by repeated freeze/thaw cycles, for example one to five cycles of freezing at a temperature of from about −30° C. to about 80° C. followed by thawing at 37° C. As another example, it can be prepared by sonification of platelet concentrates, for example for up to 30 min at a frequency of about 20 kHz. Sonification can optionally be combined with repeated freeze/thaw cycles. As another example, it can be prepared with direct platelet activation in platelet concentrates involving addition of a calcium salt (e.g., CaCl₂), activating the platelet thrombin cascade and leading to platelet lysis. Alternatively, platelet lysis can be induced by solvent/detergent treatment. In some cases, human platelet lysate preparation involves the addition to the whole blood or platelet concentrate of an anticoagulant, for example heparin. In some cases, once the human platelets have been lysed, a further centrifugation step is performed to remove platelet fragments. Human platelet lysate may be used fresh (i.e., immediately after preparation) or alternatively stored lysate may be used, for example frozen human plate lysate after thawing.

Human platelet lysate may also be obtained from commercial vendors, for example as offered by Mediatech (Manassas, VA, USA), by StemCell Technologies GmbH (Köln, Germany; Cat #06960), by Sigma-Aldrich (St Louis, MO, USA; SCM141 and SCM142), and others. In some embodiments, the hPL comprises from about 7 to about 90 mg/mL total protein.

Preferably, the human platelet lysate comprises one or more growth factors. In some embodiments, it comprises one or more growth factors selected from PDGF, PDGF-AA, PDGF-AB, PDGF-BB, TGF-beta, VEGF, bFGF, EGF, BDNF, IGF-1, and HGF, preferably selected from PDGF-AA, PDGF-AB, PDGF-BB, TGF-beta, VEGF, and IGF-1. In some embodiments, the PDGF concentration in hPL is from about 0 to 70 ng/mL, preferably from about 10 to about 70 ng/ML. In some embodiments, the PDGF-AA concentration in hPL is from about 0 to about 240 ng/mL, preferably from about 0.01 to about 240 ng/mL. In some embodiments, the PDGF-AB concentration in hPL is from about 0 to about 580 ng/mL, preferably from about 0.01 to about 580 ng/mL.

In some embodiments, the PDGF-BB concentration in hPL is from about 0 to about 23 ng/mL, preferably from about 0.01 to about 23 ng/mL. In some embodiments, the TGF-beta concentration in hPL is from about 0 to about 250 ng/mL, preferably from about 0.04 to about 250 ng/mL. In some embodiments, the VEGF concentration in hPL is from about 0 to about 0.8 ng/mL, preferably from about 0.15 to about 0.8 ng/mL. In some embodiments, the bFGF concentration in hPL is from about 0 to about 5.5 ng/mL, preferably from about 0.04 to about 5.5 ng/mL. In some embodiments, the EGF concentration in hPL is from about 0 to about 20 ng/mL, preferably from about 0.01 to about 20 ng/mL. In some embodiments, BDNF concentration in hPL is from about 0 to about 100 ng/mL, preferably from about 0.03 to about 100 ng/mL. In some embodiments, the IGF-1 concentration in hPL is from about 0 to about 500 ng/mL, preferably from about 0.1 to about 500 ng/mL. In some embodiments, the HGF concentration in hPL is from about 0 to about 2.6 ng/mL, preferably from 0.1 to about 2.6 ng/mL.

A highly preferred hPL is PLTMax Human Platelet Lysate, available from Millipore (Product Number: SCM141; Catalogue No.: 6D0352). Preferably, hPL has pH: 6.8-7.8, total protein: 4.0-6.5 g/dl, endotoxin USP: <10 EU/mL, mycoplasma: not detected; and sterility testing: negative for growth. Preferably hPL has been aseptically filtered, such as with 0.2 μm filters. These highly preferred hPLs preferably have a PDGF concentration of at least about 5 ng/mL, preferably from about 10 to about 70 ng/ML.

In some embodiments, the incubation medium comprises from 0.05 to 20 vol. % human platelet lysate, preferably from 0.2 to 10 vol. %, more preferably from 0.5 to 5 vol. %, even more preferably from 1 to 4 vol. %, most preferably from 0.5 to 2.5 vol. %, such as about 2 vol. %. In some embodiments, it comprises from 0.1 to 15 vol % human platelet lysate, preferably from 0.1 to 10 vol %, more preferably from 1 to 4 vol %, most preferably from 0.5 to 2.5 vol %, such as about 2 vol %. In preferred embodiments, the incubation medium comprises from 0.05 to 20 vol. % human platelet lysate, preferably from 0.5 to 4 vol. %, more preferably from 0.8 to 3 vol. %, even more preferably from 1 to 2.5 vol. %, most preferably from 1.2 to 2.2 vol. %, such as about 2 vol. %. In some embodiments, the incubation medium comprises 1 or about 1 vol % human platelet lysate. In preferred embodiments, the incubation medium comprises 2 or about 2 vol % human platelet lysate. In some embodiments, the incubation medium comprises 3 or about 3 vol % human platelet lysate. In some embodiments, the incubation medium comprises 4 or about 4 vol % human platelet lysate. In a preferred embodiment, the incubation medium does not comprise 1.5-2.5 vol. % or 4.5-5.5 vol. % hPL, particularly the incubation medium does not comprise or consist of α-MEM comprising 1.5-2.5 vol. % or 4.5-5.5 vol. % hPL.

The one or more samples contacted with peripheral blood mononuclear cells (PBMCs) are incubated for a duration sufficient for a PBMC, preferably a monocyte, response to be induced. In some embodiments, the duration of the incubation is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. Preferably, the duration of the incubation is at least 16 hours. In some embodiments the contacting is performed for at most 72 hours, preferably at most 36 hours, preferably at most 30 hours, more preferably at most 24 hours, still more preferably at most 18 hours, most preferably at most 16 hours.

The incubation is preferably done under cell culture, preferably human cell culture, conditions. Preferred conditions are a temperature in the range of 30-42° C. such as about 37° C. and CO₂ levels of 0-8% such as about 5%. In preferred embodiments, incubation is carried out for at least 16 hours at 37° C. and CO₂ levels of 5%. Following the incubation, the incubated sample may be used directly in step iii) or may be frozen and step iii) may be carried out at a later time point.

Step iii) Determining the Response of the PBMCs

In step iii), the response of the PBMCs, preferably mammalian PBMCs, more preferably human PBMCs, most preferably human monocytes, is determined. The response of the PBMCs can be the activation of the PBMCs. The response of the PBMCs can be the expression of a surface activation marker. Examples of surface activation markers include CD80, CD86, CD11c, CD38, CD282, and CD64. The response of the PBMCs may be the production and/or excretion, preferably excretion, of a cytokine, such as an inflammatory or anti-inflammatory cytokine, most preferably an inflammatory cytokine. Examples of inflammatory cytokines are IL-6, IL-1beta, IL-2, IL-8, IL-12, IL-17, IL-18, TNF-alpha, MCP-1, IFN-alpha, IFN-beta, IFN-gamma, and IFN-lambda, preferably are IL-6, IL-1beta, IL-8, TNF-alpha, MCP-1, more preferably IL-6. Examples of anti-inflammatory cytokines are IL-4, IL-10 IL-11, IL-13 and TGF-β. IL-6 can under some circumstances be considered to exert an anti-inflammatory role, and therefore in some embodiments IL-6 is an example of an anti-inflammatory cytokine. The response of the PBMCs can be the production and/or excretion, preferably excretion, of a prostaglandin, an example of which is PGE2. The response of the PBMCs can be the production and/or excretion, preferably excretion, of a high-mobility-group protein, an example of which is HMGB1. The response can be the production and/or excretion of neopterin. In some embodiments, the response of the PBMCs that is determined is the expression of a surface activation marker.

In some embodiments, the response of the PBMCs that is determined is the production and/or excretion, preferably excretion, of an inflammatory cytokine such as IL-6, IL-1beta, IL-8, TNF-alpha, MCP-1, IFN-alpha, IFN-beta, IFN-gamma, IFN-lambda, of a prostaglandin, or of a high-mobility-group-protein. The skilled person understands that determination of the PBMC response can also involve the combined determination of production and/or excretion, preferably excretion, of multiple inflammatory cytokines, prostaglandins, and/or high-mobility-group-proteins. In preferred embodiments, the response of the PBMCs that is determined is the production and/or excretion, preferably excretion, of IL-6.

Determination of the response of the PBMCs can be done directly after the contacting step, in the same or a different receptacle, or the incubation mixture may be stored, optionally frozen, and used for response determination at a different time point. In general, the response of the PBMCs correlates with the detection of a pyrogen. It may be that no response is detected, in which case no pyrogens are detected. Determination of the response of the PBMCs may be done, for example, with quantitative PCR, flow-cytometric techniques (such as FACS analysis), or with an immunoassay, preferably an ELISA assay. The skilled person is aware of how to perform such immunoassays, descriptions of which may be found in standard handbooks such as The Immunoassay Handbook: Theory and Applications of Ligand Binding, ELISA and Related Techniques, 2013, 4^{th} Edition, Ed. Wild, D., Elsevier Science, NL, incorporated herein by reference in its entirety. Commercial ELISA kits are also available, for example the MabTech ELISAbasic IL-6 Kit (HRP, MabTech AB, Nack Strand, SE). The ELISA assay is particularly advantageous when used in the method of the invention, as it enables high-throughput testing of multiple samples.

Accordingly, in some embodiments, the response of the PBMCs is determined by an ELISA assay. In some embodiments, the ELISA assay is performed utilizing an antibody against IL-6, IL-1 beta, IL-8, TNF-alpha, MCP-1, IFN-alpha, IFN-beta, IFN-gamma, IFN-lambda, a prostaglandin, or a high-mobility-group-protein. In preferred embodiments, the ELISA assay is performed utilizing an antibody against IL-6 (anti-IL-6). Such antibodies are commercially available, for example the clone 13A5 from MabTech AB, Nack Strand, SE. In some embodiments, the ELISA assay is performed in a standardized 96-well plate or 384-well plate, preferably a 96-well plate. An example of application of ELISA in the context of the disclosure is provided in the Examples section later herein.

In some embodiments, the response of the PBMCs is higher than the response of PBMCs in a comparative method (assay) that differs only in that human platelet serum is replaced by a different medium supplement (or in that no medium supplement is used). “Medium supplement” as used herein refers to additional medium components that can promote the response, such as activation, expression of a surface activation marker, cytokine production, prostaglandin production, or high-mobility-group protein production of PBMCs, preferably of human PBMCs, more preferably of human monocytes. Preferably, the comparative method differs only in the replacement of human platelet serum by human AB serum (hAB) or by fetal bovine serum (FBS), or in the absence of human platelet serum. Human AB serum and fetal bovine serum are used herein with their common meaning and may, for example, be obtained from commercial vendors, such as the FBS catalogue offered by VWR Avantor (Radnor, PA, USA) and the hAB offered by Merck (West Point, PA, USA).

In some embodiments wherein the pyrogen is an endotoxin, preferably a lipopolysaccharide, the response of the PBMCs is higher than the response of PBMCs in a comparative method that differs only in the replacement of the human platelet serum by human AB serum (hAB) or by fetal bovine serum (FBS), preferably by human AB serum. In some embodiments wherein the pyrogen is a non-endotoxin pyrogen, preferably a triacylated lipopeptide or a bacterial cellular component, more preferably a toll-like receptor 1/2 (TLR1/2) agonist (e.g., PAM3CSK4) or a bacterial protein (e.g., a flagellin), the response of the PBMCs is higher than the response of PBMCs in a comparative method that differs only in the replacement of the human platelet serum by human AB serum (hAB) or by fetal bovine serum (FBS), preferably by fetal bovine serum. A “higher” PBMC response may be at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, or at least 300% higher than a comparative method differing only in the replacement of human platelet serum by a different medium supplement (or in that no medium supplement is used), preferably by hAB or FBS. PBMC response may be determined as described earlier herein. For example, in embodiments wherein the determined PBMC response is IL-6 production and/or excretion, preferably excretion, a higher PBMC response means that that more IL-6 is produced and/or excreted by the PBMCs.

In some embodiments, the sensitivity of pyrogen detection of the method is higher than a comparative method that differs only in that human platelet serum is replaced by a different medium supplement (or in that no medium supplement is used), preferably by hAB or FBS. Sensitivity may preferably be expressed using the limit of quantitation (LoQ), which defines the lowest concentration of a pyrogen in a sample that is detectable by a method. A lower LoQ means that a method has increased sensitivity. The skilled person understands that the exact calculation used in LoQ determination may differ based on the assay that is used to determine the PBMC response. For example, in cases wherein a spectrophotometric immunoassay such as ELISA is used, the LoQ can be determined by identifying the first average signal (OD) of a sample that exceeds the cutoff value, which may be defined as the mean OD of the blank+10*the standard deviation of the blank, preferably determined at about 450 nm. Optionally, background OD determined at 630 nm may be subtracted from the signal before analysing. A further example of LoQ determination is provided in the Examples.

In some embodiments, the limit of quantitation of an endotoxin, preferably a lipopolysaccharide, is below 0.1 EEU/mL, preferably below 0.05 EEU/mL, more preferably below 0.02 EEU/mL, most preferably below 0.01 EEU/mL. In some embodiments, the limit of quantitation of a triacylated lipopeptide, preferably a toll-like receptor 1/2 (TLR1/2) agonist (e.g., PAM3CSK4), is below 1 ng/mL, preferably below 0.5 ng/mL, more preferably below 0.2 ng/mL, most preferably below 0.1 ng/mL. In some embodiments, the limit of quantitation of a bacterial cellular component, preferably a bacterial protein (e.g., a flagellin), is below 2 ng/mL, preferably below 1.5 ng/mL, more preferably below 1 ng/mL, most preferably below 0.5 ng/mL. Preferably, the limit of quantitation is determined in a spectrophotometric immunoassay, more preferably in ELISA, even more preferably in ELISA at about 450 nm.

In some embodiments, the LoQ of an endotoxin, preferably a lipopolysaccharide, is lower than the one exhibited by a comparative method that differs only in the replacement of the human platelet serum by human AB serum (hAB) or by fetal bovine serum (FBS), preferably by human AB serum. In some embodiments, the LoQ of a non-endotoxin pyrogen, preferably of a triacylated lipopeptide or of a bacterial cellular component, more preferably of a toll-like receptor 1/2 (TLR1/2) agonist (e.g., PAM3CSK4) or of a bacterial protein (e.g., a flagellin), is lower than the one exhibited by a comparative method that differs only in the replacement of the human platelet serum by human AB serum (hAB) or by fetal bovine serum (FBS), preferably by fetal bovine serum. A “lower” LoQ may be at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, or at least 300% lower than the one exhibited by a comparative method differing only in the replacement of human platelet serum by a different medium supplement, preferably by hAB or FBS (or in that no medium supplement is used).

The detection method of the disclosure demonstrates low variability between measurements, particularly when a plurality of identical samples is tested. Accordingly, in some embodiments, the determined response of the PBMCs for a plurality of identical samples has a coefficient of variation (ratio of the standard deviation to the mean measurement) of at most 30%. In some embodiments, the determined response of the PBMCs for a plurality of identical samples has a coefficient of variation of at most 25%, or 24%, 23%, 22%, or 21%. Preferably the determined response of the PBMCs for a plurality of identical samples has a coefficient of variation of at most 20%. More preferably the determined response of the PBMCs for a plurality of identical samples has a coefficient of variation of at most 15%. Still more preferably the determined response of the PBMCs for a plurality of identical samples has a coefficient of variation of at most 10%, most preferably at most 9.5%.

The detection method of the disclosure further demonstrates low variability between measurements of a plurality of samples of different concentrations. This variability may, for example, be determined by generating a PBMC response/pyrogen concentration curve (e.g., using a pyrogen standard as described later herein) and applying nonlinear regression analysis. Accordingly, in some cases, the concentration curve is a standard curve. Typically, a four or five parameter logistic (4PL or 5PL) regression model is applied in bioassay analysis, preferably in ELISA. Such analysis is well-known to the skilled person and can be carried out using commercial software, such as Graphpad Prism (GraphPad Software, San Diego, CA, USA). A higher quality detection method is characterized by a lower standard deviation of the measurements. It is further characterized by a higher “goodness of fit” (R²) of the 4PL or 5PL model fit to the PBMC response/pyrogen concentration curve. In addition, a higher quality method is characterized by an increased (dynamic) measurement range, which may be defined as the absolute difference between the lowest and highest values of the fit. An increased (dynamic) range can enhance the accuracy of measurements within curves with a similar slope, i.e., provide a greater concentration range in which a pyrogen may be accurately detected.

In some embodiments wherein a plurality of samples of different concentrations is provided, the goodness of fit (R²) of the method in a 4PL or 5PL, preferably 4PL, model fit is higher than of a comparative method that differs only in that human platelet serum is replaced by a different medium supplement (or in that no medium supplement is used), preferably by hAB or FBS. A higher goodness of fit may be at least 5%, at least 10%, at least 15%, at least 20%, at least 30%, at least 40%, or at least 50% higher. In some embodiments, the R² of the method in a 4PL model fit is at least 0.85, at least 0.86, at least 0.87, at least 0.88, at least 0.89, at least 0.9, at least 0.91, at least 0.92, at least 0.93, at least 0.94, at least 0.95, at least 0.96, or at least 0.98, preferably at least 0.95.

In some embodiments wherein a plurality of samples of different concentrations is provided, the (dynamic) range of the method is increased relative to a comparative method that differs only in that human platelet serum is replaced by a different medium supplement (or in that no medium supplement is used), preferably by hAB or FBS. The increase of the (dynamic) range may be at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, or at least 300%.

In some embodiments wherein a plurality of samples of different concentrations is provided, the standard deviation of the measurements obtained in the method is lower than of a comparative method that differs only in that human platelet serum is replaced by a different medium supplement (or in that no medium supplement is used), preferably by hAB or FBS. A lower standard deviation may be at least 5%, at least 10%, at least 15%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% lower. In some embodiments, the standard deviation of the method has a value of at most about 0.3, preferably at most about 0.2, more preferably at most about 0.1, most preferably at most about 0.08. The standard deviation of replicates is preferably computed by summing the square of the distance of each replicate from the mean of that set of replicates, then dividing that sum by its degrees of freedom, and then calculating the square root.

The detection method of the disclosure enables the detection of pyrogens in samples taken from various products intended for therapeutic uses or for methods of therapy, preferably therapeutic uses for or methods of therapy of humans. Examples of such products are pharmaceutical compositions, medical devices, and medical instruments as described earlier herein. If no pyrogens are detected in/on the products, they can then safely be released (cleared) for use. In this context, release can be seen as making available to a public while certifying the product adheres to certain (relevant) standards.

Accordingly, in an aspect, there is provided a method for releasing a product, preferably a pharmaceutical product, for use, the method comprising subjecting a sample derived from the product to the method for detecting a pyrogen described earlier herein. The product is preferably released only when no or low pyrogen levels are detected. This can depend on which standard is adhered to. In some embodiments, there is provided a method for releasing a pharmaceutical composition or a medical device for use, the method comprising subjecting the pharmaceutical composition or a sample derived from the medical device to the method for detecting a pyrogen as described earlier herein.

The disclosure further provides a kit of parts. Preferably, the kit is suitable for carrying out the methods of the invention. The kit may comprise a pyrogen standard of known concentration. A preferred standard is a lipopolysaccharide (LPS) standard. A standard may be a non-endotoxin pyrogen standard, preferably a standard comprising a triacylated lipopeptide or a bacterial cellular component, more preferably comprising a toll-like receptor 1/2 (TLR1/2) agonist (e.g., PAM3CSK4) or a bacterial protein (e.g., a flagellin), Pyrogen standards may be prepared according to Ph. Eur. Guidelines (Monograph 2.6.30, supra) or may be obtained already prepared from commercial supplies, for example from EDQM (European Directorate for the Quality of Medicines & Healthcare; see e.g. the Ph. Eur. Reference Standards: Orders and Catalogue offered by EDQM). Examples of pyrogen standards are provided in the Examples section later herein. A preferred kit of parts comprises a pyrogen or endotoxin standard and a vial comprising human platelet lysate, and further optionally comprises PBMCs. Preferably, the kit further comprises incubation medium, optionally in combination with the human platelet lysate.

The pyrogen standard may correspond to one or more samples. Preferably, multiple samples, each comprising a different concentration of pyrogen, allowing for the preparation of a standard curve. Exemplary standard concentrations in the case of an endotoxin, preferably LPS, are 0.5 EU/ml, 0.25 EU/ml, 0.125 EU/ml, 0.06 EU/ml, 0.03 EU/ml, 0.016 EU/ml, and 0.008 EU/ml. Multiple replicate (identical) samples may be provided, preferably at least two, more preferably at least three, most preferably at least four. The kit may comprise PBMCs, preferably monocytes, more preferably mammalian monocytes, most preferably human monocytes. Suitable PBMCs are described earlier herein. The kit may comprise one or more 96-well or 384-well plates. 384-well plates enable increased testing throughput while minimizing reagent and overall costs compared to standard pyrogen detection methods, for example compared to monocyte activation tests utilizing 96-well plates.

Accordingly, in an aspect, there is provided a kit of parts comprising a pyrogen standard, PBMCs, human platelet lysate, and one or more 96-well or 384-well plates. In some embodiments, the kit is a monocyte activation test (MAT) kit. In some embodiments, the kit further comprises incubation medium, optionally in combination with the human platelet lysate as described earlier herein. In some embodiments, the human platelet lysate is in a concentration of from 0.05 to 20 vol %, preferably from 0.2 to 10 vol %, more preferably from 0.5 to 5 vol %, even more preferably from 1 to 4 vol %, most preferably from 0.5 to 2.5 vol %, such as about 2 vol %. In some embodiments, human platelet lysate is in a concentration of from 0.1 to 15 vol %, preferably from 0.1 to 10 vol %, more preferably from 1 to 4 vol %, most preferably from 0.5 to 2.5 vol %, such as about 2 vol %.

In some embodiments, the kit is suitable for simultaneous testing of at least three distinct products, for example of distinct pharmaceutical compositions, medical devices, or medical instruments, for the presence of a pyrogen. In some embodiments, the kit is suitable for simultaneous testing of at least four distinct products. In some embodiments, the kit is suitable for simultaneous testing of at least five distinct products. In some embodiments, the kit is suitable for simultaneous testing of at least six distinct products. In some embodiments, the kit is suitable for simultaneous testing of at least seven distinct products. In some embodiments, the kit is suitable for simultaneous testing of at least eight distinct products. In some embodiments, the kit is suitable for simultaneous testing of at least nine distinct products. In some embodiments, the kit is suitable for simultaneous testing of at least ten distinct products. Testing is preferably done according to Ph. Eur. Guidelines for detection of pyrogens and endotoxins (Monograph 2.6.30, supra).

“Simultaneous” testing refers to testing of samples corresponding to the distinct products in a single plate, which allows for increased testing throughput and minimization of reagent and overall costs.

In some embodiments, the incubation medium in each sample tested per well has a volume of at most 300 μL, preferably of at most 250 μL. In some embodiments, it has a volume of about 175 μL. In some embodiments, it has a volume of at most 170, 165, 160, 155, or 150 μL. In some embodiments, the incubation medium in each sample tested per well has a volume of from 20 to 250 μL, preferably of from 30 to 175 μL, more preferably from 50 to 110 μL. In some embodiments, the incubation medium in each sample tested per well has a volume of from 20 to 150 μL, preferably of from 30 to 140 μL, more preferably of from 40 to 130 μL, more preferably of from 50 to 120 μL, more preferably of from 60 to 115 μL, more preferably of from 70 to 110 μL, more preferably of from 80 to 105 μL, more preferably of from 90 to 100 μL. In some embodiments, the incubation medium in each sample tested per well has a volume of from 20 to 100 μL, preferably of from 30 to 100 μL, more preferably of from 50 to 100 μL. In some embodiments, the incubation medium in each sample tested per well has a volume of from 80 to 120 μL. In some embodiments, the incubation medium in each sample tested per well has a volume of from 80 to 100 μL. In some embodiments, the incubation medium in each sample tested per well has a volume of 100 μL or about 100 μL.

In some embodiments, the PBMCs are present at a density of at most 500×1000 cells/cm², preferably at most 250×1000 cells cm². In some embodiments, the PBMCs are present at a density of from about 10×1000 cells/cm² to about 250×1000 cells/cm², preferably of from about 50×1000 cells/cm² to about 150×1000 cells/cm², more preferably of from about 90×1000 cells/cm² to about 130×1000 cells/cm², In some embodiments, the PBMCs are present at a density of 110×1000 cells/cm² or about 110×1000 cells/cm².

In some embodiments, the incubation medium in each sample tested per well has a volume of from 80 to 120 μL, and the PBMCs are present at a density of from about 90 to about 130×1000 cells/cm². In some embodiments, the incubation medium in each sample tested per well has a volume of from 100 or about 100 μL, and the PBMCs are present at a density of 110×1000 cells/cm² or about 110×1000 cells/cm².

Generally, the above volume can be considered the total volume present in the well. In some embodiments, incubation medium is added in such an amount that the total volume as described above is achieved.

### General Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method, respectively component as defined herein may comprise additional step(s), respectively component(s) than the ones specifically identified, said additional step(s), respectively component(s) not altering the unique characteristic. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value or 5%, preferably 1% more or less of the given value. As used herein, the term “and/or” indicates that one or more of the stated cases may occur, alone or in combination with at least one of the stated cases, up to with all of the stated cases. Various embodiments are described herein. Each embodiment as identified herein may be combined together unless otherwise indicated.

All patent applications, patents, and publications cited herein are incorporated herein by reference in their entireties. The present invention is in no way limited to the methods and materials explicitly described. The present invention is further described by the following examples which are offered for illustrative purposes only and should not be construed as limiting the scope of the invention.

### DESCRIPTION OF THE FIGURES

FIG. 1A-1D. IL-6 production in response to endotoxin (LPS) in presence of different medium supplements. PBMCs were incubated with a concentration range of LPS for 20 h in an incubator in presence of either FBS, hPL, or hAB. The production of IL-6 in presence of 1% vol. (FIG. 1A), 2% vol. (FIG. 1B), or 4% vol. (FIG. 1C) medium supplement was subsequently evaluated by ELISA. The results are shown as a mean OD of 4 replicate values+/−standard deviation. FIG. 1D displays the area under the curve for medium supplements at different concentrations.

FIG. 2A-2C. Comparison of the curve statistics of the effects of medium supplements FBS, hPL, and hAB on the production of IL-6 by PBMCs in response to endotoxin (LPS). 4 parameter logistic (4PL) nonlinear regression models were fitted on the data of FIG. 1A-1D. Shown are standard deviation (SD, FIG. 2A), (dynamic) range (FIG. 2B), and goodness of fit (R², FIG. 2C).

FIG. 3A-3D. IL-6 production in response to PAM3CSK4 in presence of different medium supplements. PBMCs were incubated with a concentration range of PAM3CSK4 for 20 h in an incubator in presence of either FBS, hPL, or hAB. The production of IL-6 in presence of 1% vol. (FIG. 3A), 2% vol. (FIG. 3B), or 4% vol. (FIG. 3C) medium supplement was subsequently evaluated by ELISA. The results are shown as a mean OD of 4 replicate values+/−standard deviation. FIG. 3D displays the area under the curve for medium supplements at different concentrations.

FIG. 4A-4C. Comparison of the curve statistics of the effects of medium supplements FBS, hPL, and hAB on the production of IL-6 by PBMCs in response to PAM3CSK4. 4 parameter logistic (4PL) nonlinear regression models were fitted on the data of FIG. 3A-3D. Shown are standard deviation (SD, FIG. 4A), (dynamic) range (FIG. 4B), and goodness of fit (R², FIG. 4C).

FIG. 5A-5D. IL-6 production in response to flagellin B.S. in presence of different medium supplements. PBMCs were incubated with a concentration range of flagellin B.S. for 20 h in an incubator in presence of either FBS, hPL, or hAB. The production of IL-6 in presence of 1% vol. (FIG. 5A), 2% vol. (FIG. 5B), or 4% vol. (FIG. 5C) medium supplement was subsequently evaluated by ELISA. The results are shown as a mean OD of 4 replicate values+/−standard deviation. FIG. 5D displays the area under the curve for medium supplements at different concentrations.

FIG. 6A-6C. Comparison of the curve statistics of the effects of medium supplements FBS, hPL, and hAB on the production of IL-6 by PBMCs in response to flagellin B.S. 4 parameter logistic (4PL) nonlinear regression models were fitted on the data of FIG. 5A-5D. Shown are standard deviation (SD, FIG. 6A), (dynamic) range (FIG. 6B), and goodness of fit (R², FIG. 6C).

FIG. 7A-7D. FBS induces IL-6 response against NEPs at lower percentages. PBMCs were incubated with a concentration range of endotoxin (LPS, FIG. 7A), PAM3CSK4 (FIG. 7B) or flagellin B.S. (FIG. 7C) for 20 h in an incubator in presence of FBS at different concentrations. FIG. 7D displays the area under the curve for the different pyrogens and concentrations.

FIG. 8A-8D. hPL induces IL-6 response against pyrogens. PBMCs were incubated with a concentration range of endotoxin (FIG. 8A), PAM3CSK4 (FIG. 8B) or Flagellin B.S. (FIG. 8C) for 20 h in an incubator in presence of hPL at different concentrations. The production of IL-6 was subsequently evaluated by ELISA. The results are shown as a mean OD of 4 replicate values+/−standard deviation. FIG. 8D displays the area under the curve for the different pyrogens and concentrations.

FIG. 9A-9D. Effect of cell density on IL-6 production in the monocyte activation test. PBMCs were seeded in 384-well plates at indicated cell densities (×1000 cells per cm²), in a total volume of 33 μL (FIG. 9A), 50 μL (FIG. 9B), 66 μL (FIG. 9C), or 100 μL (FIG. 9D), X-axis indicates cell density (×1000 cells per cm²). y-axis indicates IL-6 per 1000 cells. Plotted are 4 replicates (symbols) and average (horizontal line). Figure titles show total MAT volume.

FIG. 10. Plots of absorbance values in optical density (OD) (y-axis) at each concentration of LPS in EU/ml (x-axis) for each density (×1000 cells/cm², increasing from left to right). Dotted line indicates an 0.1 OD. Depicted are the averages of three experiments with four replicates each.

FIG. 11. Signal to noise ratios (bars) plotted against density (1000 cells/cm²). Signal-to-noise ratios were calculated by dividing the OD at 0.016 EU/ml by the OD at blank.

FIG. 12. Coefficient of variation (CV) at various cell densities. Average CV % of four replicates was calculated for each concentration LPS (EU/ml) and subsequently averaged per density (1000 cells/cm²). Plots show the average (bars) and standard deviation (error bars) of three different experiments.

FIG. 13. Relative gain (y-axis) plotted as the percentage of optical density (OD) at 0.032 EU/ml LPS normalized to the optical density at 100 microliter assay volume. X-axis denotes the cell density in 1000 cells/cm². Error bars represent the standard deviation of three experiments.

FIG. 14. Average CV % at different assay volumes. Average CV % of four replicates was calculated for each concentration LPS (EU/ml) and subsequently averaged per assay volume (microliter) and density. Plots show the average (bars) and standard deviation (error bars) of three different experiments (from left to right, 55/110/220). Patterns denote cell densities in 1000 cells/cm².

FIG. 15. Absorbance (OD) plotted against concentration LPS (EU/ml). X-axis is in log scale. Gray line represents a standard curve at 110 density (×1000 cells/cm²) and in 100 microliter assay volume. Black line represents a standard curve at 220 density (×1000 cells/cm²) and in 66 microliter assay volume. Error bars indicate standard deviation of four replicates.

FIG. 16. Curve slope. Bars indicate curve slope of four-parameter logistic curve. Left: LPS standard curve at 110 density (×1000 cells/cm²) and 100 microliter assay volume. Right: LPS standard curve at 220 density (×1000 cells/cm²) and 66 microliter assay volume.

FIG. 17. Average CV %. The average CV % of four replicates was calculated for each concentration LPS (EU/ml) and subsequently averaged per volume/density combination. Left: Average CV % of LPS standard curve at 110 density (×1000 cells/cm²) and 100 microliter assay volume. Right: Average CV % of LPS standard curve at 220 density (×1000 cells/cm²) and 66 microliter assay volume.

### EXAMPLES

### Example 1. Human Plate Lysate Results in Enhanced Reactivity Towards Endotoxins and Non-Endotoxin Pyrogens

### PBMC Handling

PBMCs (10 million PBMCs/ml) were rapidly thawed in a water bath (Grant JB Nova, Cambridge, UK) set to 37° C. and resuspended by slowly adding (˜1 mL per 5 sec) pre-warmed (37° C.) RPMI 1640+Glutamax+HEPES (Gibco, Grand Island, NY, USA) medium. Cells were immediately added to plates containing medium supplements: human Platelet Lysate (hPL, PLTMax Human Platelet Lysate, available from Millipore (Product Number: SCM141; Catalogue No.: 6D0352)), Fetal Bovine Serum (FBS, Avantor by VWR, USA) or Human AB (hAB, Merck, West Point, PA, USA) serum. Each sample well contained about 1 million PBMCs/ml, corresponding to a density of about 300×1000 cells/cm².

### Preparations LPS, PAM3CSK4, and Flagellin B.S

Lipopolysaccharide (LPS) was obtained from the European Directorate for the Quality of Medicines & HealthCare (EDQM, batch 5.1), and handled as instructed by the EDQM. Briefly, the LPS was rehydrated by vortex mixing for 30 min in 5 mL LAL reagent water (LRW, Lonza Bioscience, Basel, Switzerland) and diluted by vortex mixing for 3 min in LRW to a stock concentration of 100 (EEU/mL). LPS reference endotoxin (RSE) samples were prepared in RPMI 1640+Glutamax+HEPES (Gibco, Grand Island, NY, USA) and mixed by resuspension for 20 times.

PAM3CSK4 (a toll-like receptor 1/2 (TLR1/2) agonist) (Invivogen, Toulouse, France) stock solution (10 μg/mL) was created by adding 950 microliters of RPMI 1640+Glutamax (Gibco, Grand Island, NY, USA) to 50 microliter aliquots of 10 μg/mL and mixed by vortexing briefly. The stock solution was diluted to a sample concentration of 50 ng/mL, after which a 2-fold serial dilution (2 mL+2 mL) was created by resuspension in RPMI. 50 μL of each dilution mastermix was added to the plate, keeping the concentration equal for each medium supplement tested.

Flagellin-BS (Invivogen) stock solution (500 ng/mL) was created by adding 950 μL of RPMI to 50 microliter aliquots of 10 μg/mL and mixed by resuspension. The stock solution was diluted to a sample concentration of 125 ng/mL, after which a 2-fold serial dilution (2 mL+2 mL) was created by resuspension in RPMI. 50 μL of each dilution mastermix was added to the plate, keeping the concentration equal for each medium supplement tested.

### ELISA

ELISA plates (MaxiSorp, NUNC, Amsterdam, the Netherlands) were coated with IL-6 capture antibody (clone 13A5, Mabtec) at a 1:2000 concentration diluted in PBS (VWR, Solon, OH, USA) and incubated overnight at 4° C. ELISAs were performed according to the manufacturer's protocol (MabTech AB, Nacka Strand, Sweden). Optical density (OD) was measured using an absorbance microplate reader Multiscan Ascent (Thermo scientific, Vantaa, Finland) at a wavelength of 450 nm. Background OD at 630 nm was subtracted from the 450 nm signal before further analysing. Supernatant (50 μL) was diluted 1:4 in ELISA diluent (PBST (ELISA wash buffer, Biolegend, Amsterdam, The Netherlands)+0.1% BSA) and added to the ELISA microplates.

### Statistics

Statistical analysis including 4PL logistic regression was performed using Graphpad Prism 8.

### Results

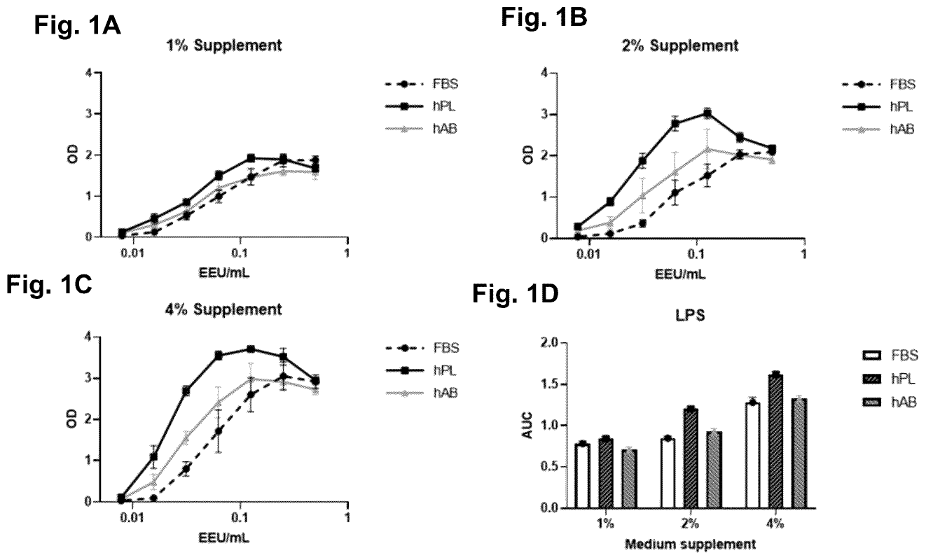

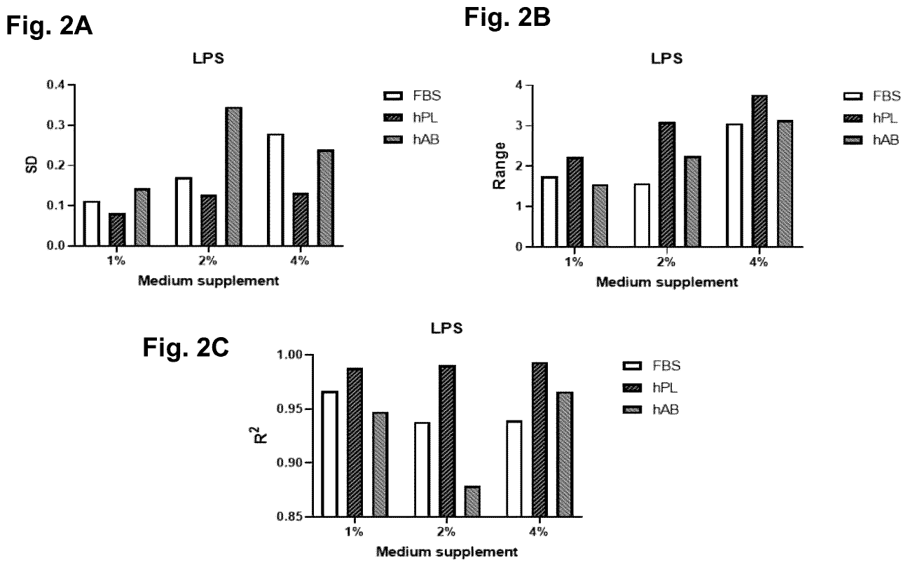

To investigate whether hPL can be used to improve pyrogen detection using MAT, we incubated a pool of PBMCs from four single donors with an endotoxin concentration range and subsequently examined IL-6 production by measuring ELISA absorbance in optical density (OD) (FIG. 1A-1D) after 20 hours using hPL, FBS, and hAB as medium supplements. The results show a dose-dependent IL-6 production for all medium supplements that is elevated with increasing vol. percentage of medium supplement (FIG. 1A-1C). Of all medium supplements, hPL consistently displayed a higher LPS response compared to the other medium supplements (FIG. 1D), while LPS incubation in presence of FBS and hAB induced a similar IL-6 production across percentages tested. To further investigate the effects of medium supplements on the IL-6 response of PBMCs to LPS, we fitted 4 parameter logistic (4PL) nonlinear regression models on the curves of FIGS. 1A-1C and compared the statistical parameters standard deviation (FIG. 2A), (dynamic) range (FIG. 2B), and goodness of fit (R², FIG. 2C). A 4PL logistic regression model is a type of nonlinear regression commonly used in dose-response curves e.g., in the MAT and/or receptor-binding analysis that requires four parameters to fit an S-shaped curve. From this model, interpolation of sample data can be performed to determine the concentration of a contamination, expressed in EEU/mL, in a sample to be tested in the MAT. Therefore, the quality of this 4PL logistic regression model fitted on the reference endotoxin standard (RSE) curve is essential for accurately determining the sample contaminant level. A high quality 4PL regression model is characterized by a low variation (standard deviation), an increased range, and a high goodness of fit (R²) of the model to the data of the RSE. The results of the 4PL regression show a consistently lower standard deviation (SD) for hPL compared to the other medium supplements (FIG. 2A), indicating reduced variation across all percentages tested. Moreover, the range of hPL was consistently increased when hPL was used compared to the other supplements (FIG. 2B), indicating an enhanced dynamic range to determine IL-6 production. The goodness of fit (R²) was also higher (FIG. 2C) for hPL compared to the other supplements. These results indicate that hPL shows the strongest response with less variation, more range and enhanced goodness of fit compared to the other supplements in detecting LPS.

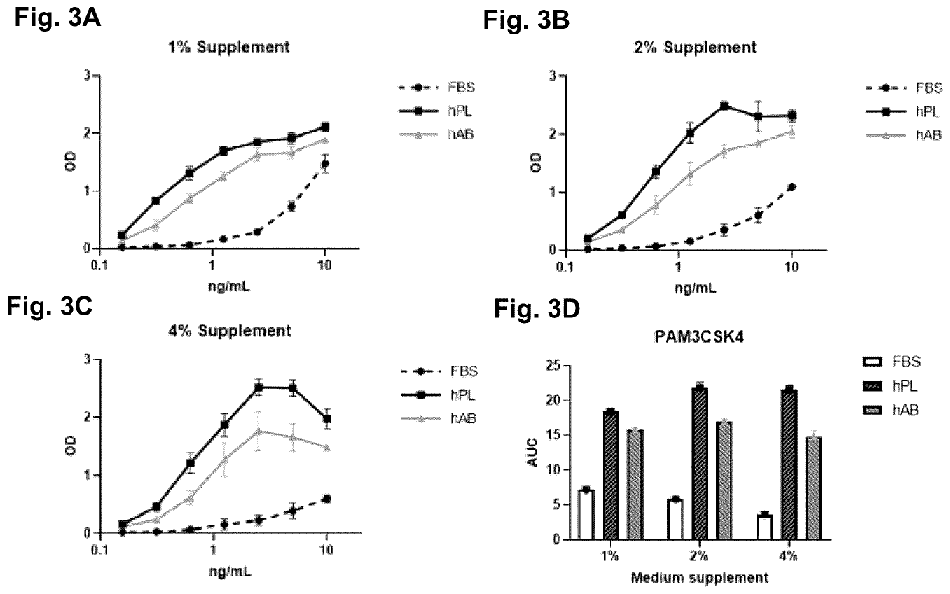

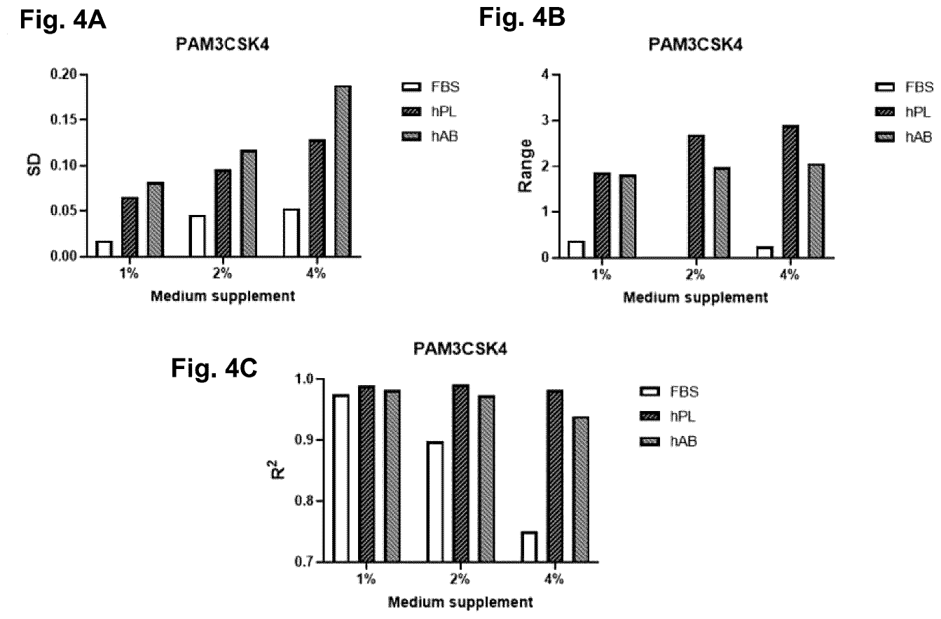

Expanding on the results for endotoxin, the effects of medium supplements hPL, FBS, and hAB on the ability of the MAT to determine the presence of toll-like receptor (TLR)-1/2 ligands was investigated. To this end, a pool of PBMCs from four single donors was incubated with a concentration range of PAM3CSK4 and examined for IL-6 production by ELISA (FIG. 3A-3D). Both human-based medium supplements induced a strongly enhanced IL-6 response to PAM3CSK4 compared to FBS (FIG. 3A-3D), indicating an increased ability to determine TLR-1/2 ligands for human based over animal based supplements. Similar to LPS, hPL displayed the highest IL-6 response to PAM3CSK4 across all percentages tested, with an increased area under the curve compared to the other medium supplements. Comparing the statistics of (4PL) nonlinear regression models fitted on the data of FIG. 3A-3C revealed a lower SD for FBS over the other supplements (FIG. 4A), possibly exaggerated by the lower overall OD values for FBS over human-based supplements. Of the human-based supplements, hPL displayed a consistently lower SD compared to hAB, again indicating reduced variation across vol. percentages tested (FIG. 4A). Furthermore, hPL displayed an increased range (FIG. 4B), and an improved goodness of fit (FIG. 4C) compared to the other medium supplements. These results indicate that human-based supplements are superior to FBS for the detection of TLR-1/2 ligands, among which hPL displays reduced variation, improved goodness of fit and a higher range compared to hAB.

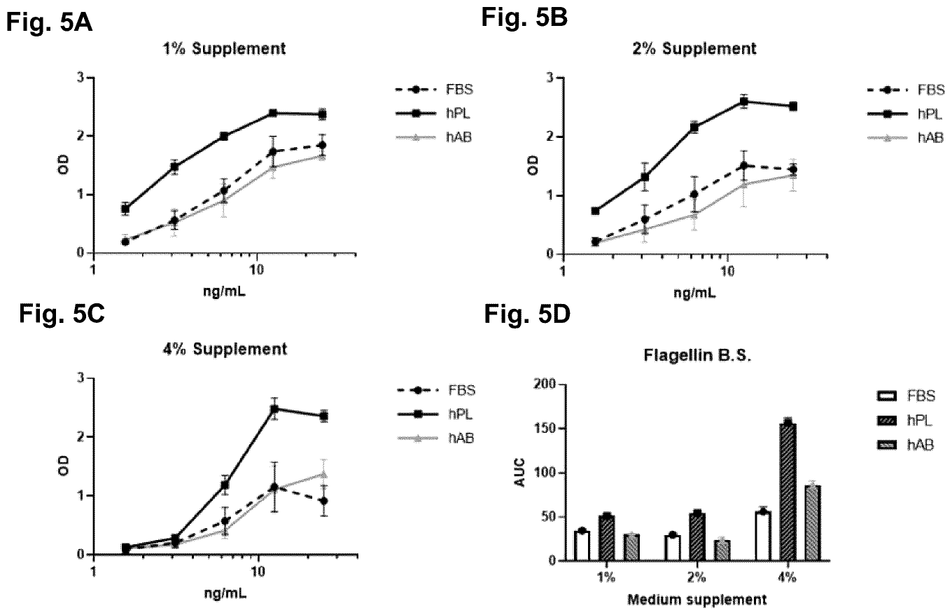

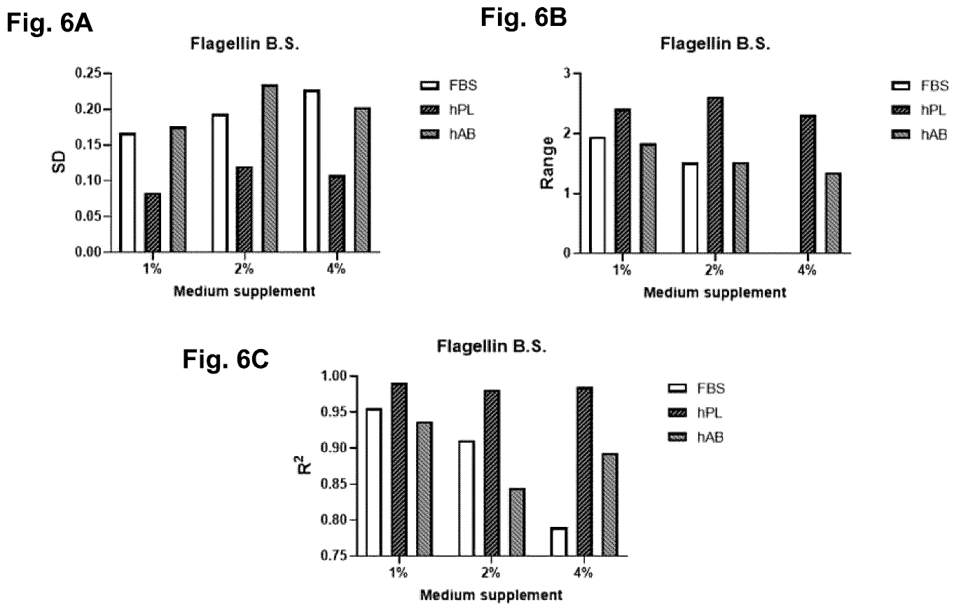

In addition to endotoxin and PAM3CSK4, the effects of medium supplements hPL, FBS, and hAB on the IL-6 response to the common NEP Flagellin from Bacillus Subtilis (Flagellin B.S.) was investigated. Again, a pool of PBMCs from four different donors was incubated with a concentration range of Flagellin B.S. after which the production of IL-6 was determined by ELISA. Across all vol. percentages tested, hPL induced the strongest IL-6 production in response to Flagellin B.S. with an enhanced area under the curve compared to all other medium supplements (FIG. 5A-5D), indicating an increased ability to detect Flagellin B.S. for hPL. A comparison of the (4PL) nonlinear regression models fitted on the data of FIG. 5A-5C, showed a consistently reduced SD (FIG. 6A), increased range (FIG. 6B) and improved goodness of fit (FIG. 6C) for hPL over the other medium supplements. These results indicate that hPL is a superior medium supplement for the detection of Flagellin B.S. compared to hAB as well as FBS.

The effects of medium supplements hPL, FBS, and hAB on the Limit of Quantitation (LoQ), a stringent measure of sensitivity, of the MAT to endotoxin, PAM3CSK4 and Flagellin B.S. were compared. The LoQ was determined by identifying the first average signal (OD) that exceeded the cutoff value, defined as the mean OD of the blank+10*the standard deviation of the blank. Of all medium supplements tested, the LoQ of hPL was consistently lower compared to the other medium supplements for all pyrogens included in the study compared to FBS (Table 1). Specifically, the LoQ of hPL for PAM3CSK4 was 0.9 and 0.04 ng/mL lower than FBS and hAB, respectively. Moreover, the LoQ of hPL for Flagellin B.S. was 0.4 and 0.8 lower than FBS and hAB, respectively. Finally, the LoQ of the human based supplements for LPS was 0.008 EEU/mL lower than FBS, but equal compared to each other. These observations indicate that hPL is the most consistent of the medium supplements in the detection of samples containing multiple types of pyrogens, with a relatively high sensitivity across all pyrogens included in the study.

TABLE 1
Limit of Quantitation (LoQ) of the MAT, using 2%
FBS, 2% hPL or 2% hAB as a medium supplement.
Limit of quantitation (LoQ)

| | LPS | PAM3CSK4 | Flagellin B.S |
|---|---|---|---|
| | EEU/mL | ng/mL | ng/mL |
| FBS | 0.016 | 0.131 | 0.8 |
| hPL | 0.008 | 0.04 | 0.4 |
| hAB | 0.008 | 0.08 | 1.6 |

### Example 2. Determination of Optimal Medium Supplement % Vol. For MAT

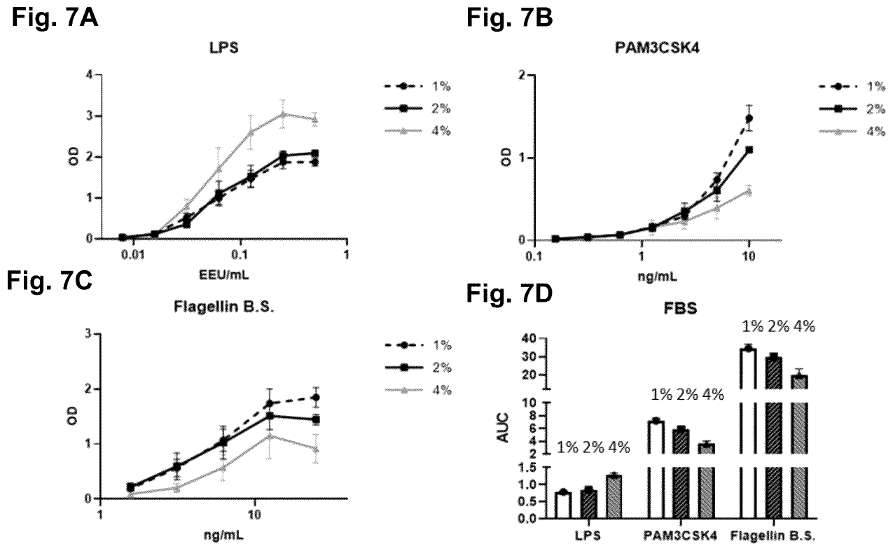

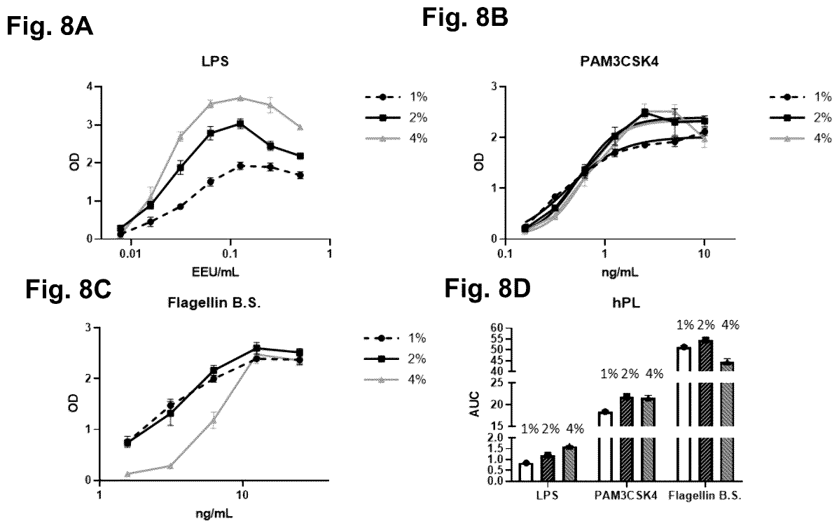

The data of FIGS. 1A-1C, 3A-3C, and 5A-5C for FBS were combined (FIG. 7A-7D) to compare the LPS, PAM3CSK4, and Flagellin B.S. response between different vol. percentages. From the data, it can be observed that there is a dose-response relationship between FBS and IL-6 production for LPS (FIG. 7A). However, lower vol. percentages of 1% and 2% FBS induce an increased IL-6 response to NEPs compared to 4% (FIGS. 7B & C), while it is still possible to detect LPS at those lower percentages (FIG. 7A). For this reason, the use of 1% and 2% FBS is superior to 4% FBS. The data of FIGS. 1A-1C, 3A-3C, and 5A-5C for hPL were also combined (FIG. 8A-8D) to compare the LPS, PAM3CSK4, and Flagellin B.S. response between different vol. percentages. Similarly to FBS, a dose-response relationship between hPL and IL-6 production can be observed for LPS (FIG. 8A). Further, a vol. percentage of 2% induced an increased IL-6 response to NEPs compared to 4% (FIG. 8D).

### Example 3. Optimization of Cell Density and Assay Volume

### Control Preparations

Lipopolysaccharide (LPS) was obtained from EDQM (batch 5.1), and handled as instructed by the EDQM. The LPS was rehydrated by vortex mixing for 30 min in 5 mL LAL reagent water (LRW, Lonza Bioscience, Basel, CH) and diluted by vortex mixing for 3 min in LRW to a stock concentration of 10 endotoxin units per milliliter (EU/ml). LPS reference endotoxin curves (RSE) were subsequently created via serial dilution, mixed by resuspension in RPMI 1640 (Thermo-Fisher Scientific, Waltham, MA, USA).

### PBMC Handling

Vials of PBMCs (10 million PBMCs/ml) were rapidly thawed in a water bath set to 37° C. and resuspended by slowly adding pre-warmed (37° C.) RPMI medium containing 4% human medium supplement (Mediatech, Manassas, VA, USA).

### Cell Density

Samples of 0.2 EU/ml LPS were plated onto a 384-well microplate (Thermo-Fisher Scientific, Waltham MA, USA) at 50% of the final volume for various different final volumes as indicated (per experiment). Subsequently, cell suspensions were added in a 1:1 ratio at different cell concentrations as indicated (per experiment), to obtain a final concentration of human media supplement (HMS) of 2% (vol./vol.). Final concentrations of LPS corresponds to a two-fold dilution series starting at 0.1 EU/mL, obtained by resuspension in RPMI in the plate. The cells were incubated with the LPS for 20 hours+/−1 hour in an incubator (Binder (CB60), Tuttlingen, Germany) set to 37° C. and 5% CO₂, after which IL-6 concentrations were measured by ELISA as explained below. The IL-6 per 1000 cells was calculated by interpolating the measured optical density (OD) on a linear regression model of an IL-6 standard curve and dividing total IL-6 produced by the total number of cells in the well.

LPS standard curves (33 microliters at concentrations of 0.064 EU/ml, 0.032 EU/ml, 0.016 EU/ml, 0.008 EU/ml, 0.004 EU/ml) were plated onto a 384-well microplate. Cryopreserved peripheral blood mononuclear cells (PBMCs) were thawed and resuspended in RPMI medium containing 4% human medium supplement. Cell suspensions were serially diluted (dilution factor 2) at concentrations of 1514 cells per microliter, 757 cells per microliter, 378 cells per microliter, 189 cells per microliter, 94.7 cells per microliter, and 47.4 cells per microliter. 33 microliters of each cell suspension was added to the plate, resulting in a final cell density of approximately 440.000 cells/cm², 220.000 cells/cm², 110.000 cells/cm², 55.000 cells/cm², 27.500 cells/cm², and 13.700 cells/cm², respectively. Final concentrations of LPS were 0.032 EU/ml, 0.016 EU/ml, 0.008 EU/ml, 0.004 EU/ml, 0.002 EU/ml at each density. Final concentration of HMS was 2% in each well.

### MAT Incubation Volume

Samples for LPS standard curves (concentrations: 0.064 EU/ml, 0.032 EU/ml, 0.016 EU/ml, 0.008 EU/ml, 0.004 EU/ml) were plated onto a 384-well microplate at three different volumes (16.7 microliters, 33 microliters, and 50 microliters). Cryopreserved PBMCs were thawed and reconstituted in RPMI medium containing 4% human medium supplement. Cell suspensions were diluted at different cell concentrations, and added to the plate in a 1:1 ratio, resulting in final cell densities of 55.000 cells/cm², 110.000 cells/cm² and 220.000 cells/cm² at each assay volume (33 microliters, 66 microliters and 100 microliters), at a final HMS concentration of 2%.

### MAT

Samples for LPS standard curves were added to the culture plate at different volumes (in a 1:1 ratio with resuspended PBMCs and incubated for 16 hours in an incubator with 5% CO₂ at 37° C. Final concentrations of LPS were 0.5 EU/ml, 0.25 EU/ml, 0.125 EU/ml, 0.06 EU/ml, 0.03 EU/ml, 0.016 EU/ml and 0.008 EU/ml. Final concentration of HMS was 2%.

### ELISA

ELISA plates were coated with IL-6 capture antibody (clone 13A5, MabTech AB, Nack Strand, SE) at a 1:2000 concentration diluted in phosphate-buffered saline and incubated overnight at 4° C. ELISA was performed using the MabTech ELISAbasic IL-6 Kit (HRP) according to the manufacturer's protocol (MabTech). Absorbance was measured using a Thermo-Scientific absorbance plate reader at a wavelength of 450 nanometers. Background at 630 nanometers was subtracted. Supernatant (16.7 microliters) was diluted 1+1 in incubation buffer and added to the ELISA plate.

### Statistics

Statistical analysis including four-parameter/five-parameter logistic regression was performed using Graphpad Prism 8 (GraphPad Software, San Diego, CA, USA).

### Results

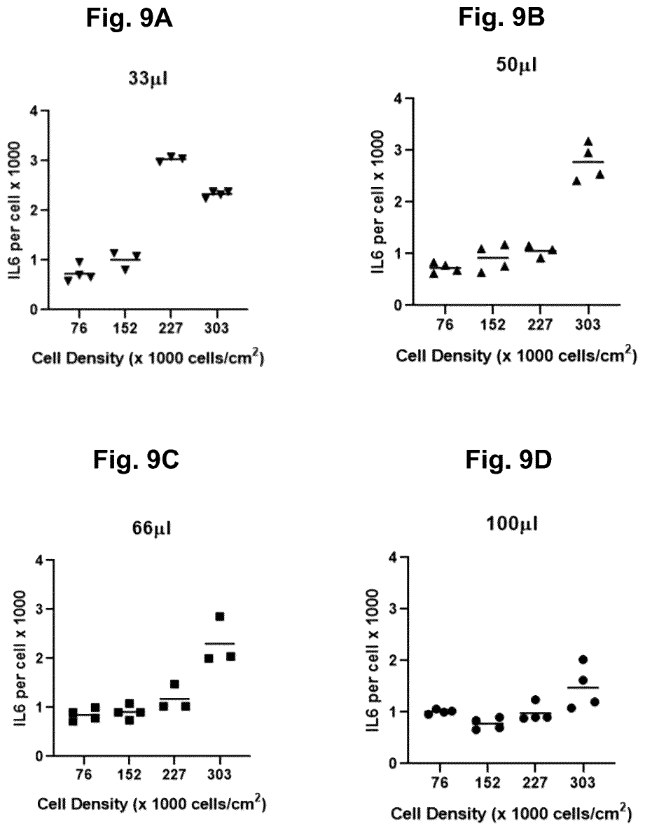

Increasing cell density showed improved IL-6 response per 1000 cells at different 384 MAT volumes at a concentration of 0.1 EU/ml, with sharp increases seen at 303 density (×1000 cells/cm²) in 50 μL, 66 μL, and 100 μL. Using 33 μL volume in the MAT, a 3-fold increase in signal was seen at 227 density (×1000 cells/cm²), compared to 152 density (×1000 cells/cm²) while a slight decrease in IL-6 per cell was seen at 303 density (×1000 cells/cm²) (FIG. 9A-9D).

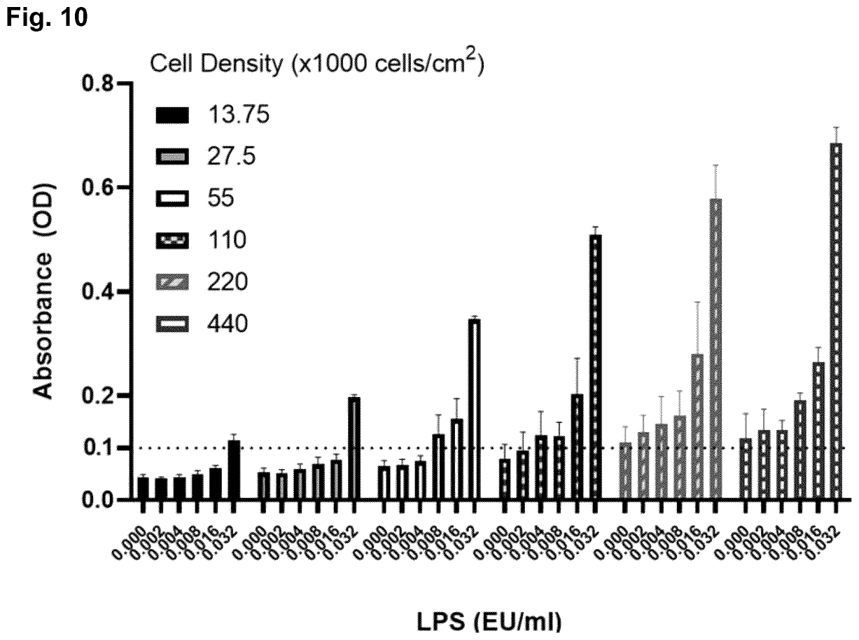

Higher densities inferred stronger LPS signal (OD) at each LPS concentration but also increased background signal (at 0.00 EU/ml) up to 0.12 OD at 440 density (×1000 cells/cm²) (FIG. 10). In addition, signal-to-noise ratio as calculated by signal at 0.032 EU/ml divided by background signal, increased when increasing density with an optimum reached at 110 density (×1000 cells/cm²) (signal-to-noise 6.4).

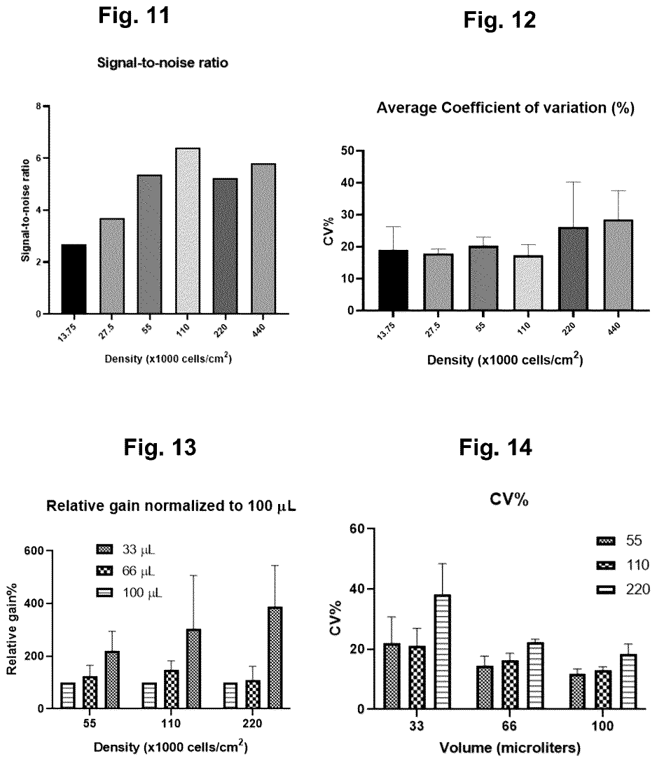

Furthermore, while LPS signal and signal to noise ratio increases with density (FIG. 11), replicate variation also increases as shown in FIG. 12, with an average CV % up to 28.5% at 440 density (×1000 cells/cm²). In between 55 and 220 density (×1000 cells/cm²) the signal-to-noise ratio was the highest among the densities tested, while the average CV % was the lowest.

Thirty-three microliters of MAT volume showed significant increase in LPS response, as depicted in FIG. 13, showing the relative gain at 0.032 EU/ml normalized to 100 μL. Relative gain also increased when plating higher cell densities with an average relative gain of 219% at 55 density (×1000 cells per cm²), 304% at 110 density (×1000 cells per cm²), and 387% at 220 density (×1000 cells per cm²) at MAT volumes of 33 μL. However, smaller volumes showed increased variation between replicates (FIG. 14). At each density, 100 μL MAT volume showed the lowest CV % (11.8% at 55 density (×1000 cells per cm²), 13.3% at 110 density (×1000 cells per cm²) and 18.6% at 220 density (×1000 cells/cm²)). A pattern of increased CV % with increased density was observed for each assay volume, with the exception of 110 density (×1000 cells per cm²), which showed a lower CV % than 220 density (×1000 cells per cm²) in all tested assay volumes.

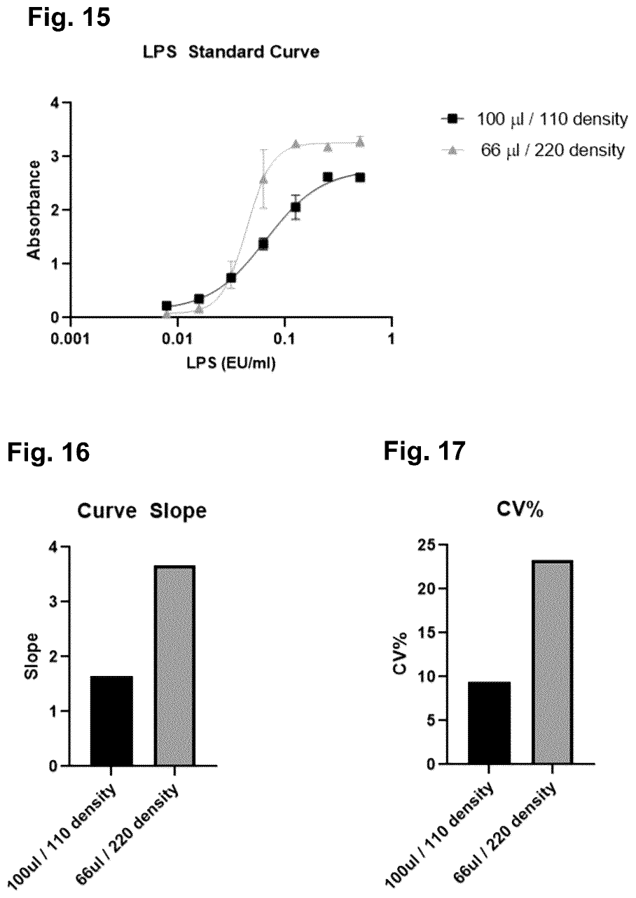

For two configurations: MAT volume at 66 μL with a cell density of 220 (×1000 cells per cm²) and MAT volume of 100 μL with a cell density of 110 (×1000 cells per cm²), an LPS standard curve was created (FIG. 15). Four-parameter logistic regression showed R² values of 0.99 (66 μL/220 density (×1000 cells per cm²)) and 0.98 (100 μL/100 density (×1000 cells per cm²)). FIG. 16 shows the curve slopes of both graphs, with approximately two-fold difference in slope between the two configurations. In addition, average CV % was markedly higher in the 66 μL/220 density (×1000 cells per cm²) MAT than the 100 μL/110 density (×1000 cells per cm²) (23.4% and 9.4%, respectively) (FIG. 17).

1. Method for detecting a pyrogen in a sample, the method comprising the steps of:
i) providing one or more samples;
ii) contacting the sample with peripheral blood mononuclear cells (PBMCs) in an incubation medium comprising human platelet lysate (hPL); and
iii) determining the response of the PBMCs.

2. The method according to claim 1, wherein the method is a monocyte activation test.

3. The method according to claim 1 or 2, wherein the incubation medium has a volume of at most 300 μL or at most 250 μL per sample, preferably wherein the incubation medium has a volume of from 20 to 250 μL, more preferably of from 30 to 175 μL, most preferably of from 50 to 110 μL.

4. The method according to any one of claims 1-3, wherein the contacting of step ii) is performed in a standardised 96-well plate or 384-well plate, preferably a 96-well plate.

5. The method according to any one of claims 1-4, wherein the incubation medium comprises from 0.05 to 20 vol. % human platelet lysate, preferably from 0.5 to 4 vol. %, more preferably from 0.8 to 3 vol. %, even more preferably from 1 to 2.5 vol. %, most preferably from 1.2 to 2.2 vol. %, such as about 2 vol. %.

6. The method according to any one of claims 1-5, wherein the response of the PBMCs that is determined is the excretion of an inflammatory cytokine such as IL-6, IL-1 beta, IL-8, TNF-alpha, MCP-1, IFN-alpha, IFN-beta, IFN-gamma, IFN-lambda, of a prostaglandin, or of a high-mobility-group-protein.

7. The method according to claim 6, wherein the response of the PBMCs is higher than the response of PBMCs in a comparative method that differs only in the replacement of human platelet serum by human AB serum or by fetal bovine serum, or in the absence of human platelet serum.

8. The method according to any one of claims 1-7, wherein the response of the PBMCs is determined by an ELISA assay.

9. The method according to any one of claims 1-8, wherein the PBMCs are present at a density of at most 500×1000 cells/cm², preferably at most 250×1000 cells cm².

10. The method according to any one of claims 1-9, wherein the PBMCs are present at a density of from about 10×1000 cells/cm² to about 350×1000 cells/cm², preferably of from about 50×1000 cells/cm² to about 150×1000 cells/cm², more preferably of from about 90×1000 cells/cm² to about 130×1000 cells/cm².

11. The method according to any one of claims 1-10, wherein the limit of quantitation of lipopolysaccharides is below 0.01 EEU/mL, and/or the limit of quantitation of triacylated lipopeptide is below 0.1 ng/mL, and/or the limit of quantitation of bacterial protein is below 1 ng/mL.

12. The method according to any one of claims 1-11, wherein in step iii) the response is determined for a plurality of samples, wherein the response of the PBMCs for the plurality of identical samples has a coefficient of variation of at most 30%.

13. The method according to any one of claims 1-12, wherein the incubation medium has a volume of from about 80 to about 120 μL, and wherein the PBMCs are present at a density of from about 90 to about 130×1000 cells/cm².

14. The method according to any one of claims 1-13, wherein the incubation medium has a volume from 30 to 175 μL, most preferably of from 50 to 110 μL.

15. The method according to any one of claims 1-14 wherein the PBMCs are present at a density of from about 50×1000 cells/cm² to about 150×1000 cells/cm², more preferably of from about 90×1000 cells/cm² to about 130×1000 cells/cm².

16. The method according to any one of claims 1-15 wherein the PBMCs are non-immortal PBMCs.

17. The method according to any one of claims 1-16, wherein the incubation medium has a volume from 30 to 175 μL, and wherein the PBMCs are present at a density of from about 50×1000 cells/cm² to about 150×1000 cells/cm².

18. The method according to any one of claims 1-17, wherein the incubation medium comprising hPL is RPMI (Roswell Park Memorial Institute) medium, DMEM (Dulbecco's Modified Eagle's Medium), EMEM (Eagle's Minimum Essential Medium), Ham's F-10 or F-12 medium, or Iscove's Modified Dulbecco's Medium (IMDM).

19. Method for releasing a pharmaceutical composition or a medical device for use, the method comprising subjecting the pharmaceutical composition or a sample derived from the medical device to a method as described in any one of claims 1-18.

20. Kit of parts comprising a pyrogen or endotoxin standard and a vial comprising human platelet lysate, further optionally comprising PBMCs.
